(19) United States
(12) Patent Application Publication
(10) Pub. No.: US 2005/0176683 A1
(43) Pub. Date: Aug. 11, 2005

(54) Methodology for preparing combinatorial libraries based upon a bicyclic scaffold

(76) Inventors: David G. Hangauer, East Amherst, NY (US); Mohamed Ayman Al-Zahaby, Cairo (EG)

Correspondence Address: Michael L. Goldman;Nixon Peabody LLP, Clinton Square, P.O. Box 31051, Rochester, NY, 14603-1051, US

(21) Appl. No.: 11/005,098

(22) Filed: Dec. 6, 2004

Related U.S. Application Data
(60) Provisional application No. 60/526,854, filed on Dec. 4, 2003.

(51) Int. Cl. G01N 33/53; A61K 31/69; A61K 31/675; A61K 31/4188; C07D 487/04

(52) U.S. Cl. 514/64; 514/393; 435/7.1; 436/518; 548/303.1; 548/112; 514/80

(57) ABSTRACT

The present invention relates to tetrahydro-pyrrolo[1,2-a]imidazole-2,5-dione derivative compounds, hexahydro-pyrrolo[1,2-a]pyrimidine-2,6-dione derivative compounds, and combinatorial libraries comprising such compounds. The present invention also relates to methods of preparing such compounds. The methods first involve providing a functionalized resin solid support. Next, the functionalized resin solid support is reacted with amino acids under conditions effective to produce a resin bound dipeptide or tripeptide alcohol intermediate compound. Then, the resin bound dipeptide or tripeptide alcohol intermediate compound is oxidized under conditions effective to convert the resin bound dipeptide or tripeptide alcohol intermediate compound to a resin bound dipeptide or tripeptide aldehyde intermediate compound. Finally, the resin bound dipeptide or tripeptide aldehyde intermediate compound is cyclized under conditions effective to produce the tetrahydro-pyrrolo[1,2-a]imidazole-2,5-dione derivative or hexahydro-pyrrolo[1,2-a]pyrimidine-2,6-dione derivative compound. The methods can be performed in solid phase, solid/solution phase, or solution phase.

This application claims the benefit of U.S. Provisional Patent Application Ser. No. 60/526,854, filed Dec. 4, 2003, which is hereby incorporated by reference in its entirety.

### FIELD OF THE INVENTION

The present invention relates to tetrahydro-pyrrolo[1,2-a]imidazole-2,5-dione derivative compounds, hexahydro-pyrrolo[1,2-a]pyrimidine-2,6-dione derivative compounds, combinatorial libraries of such compounds, and methods of preparing such compounds.

### BACKGROUND OF THE INVENTION

There are estimated to be approximately 10¹⁰⁰ potential drug-sized (i.e., molecular weight of 500 or less) molecules. This enormous number makes it impossible to prepare and test all drug-sized molecules. Most large libraries of single purified compounds that have been prepared to date are based upon very simple scaffolds, e.g., aromatic scaffolds with no stereochemistry, because the synthesis of these simple scaffold libraries tends to be easier. With a large global effort to prepare libraries for drug lead screening, the simple scaffold libraries are rapidly being milked out for potential drug leads. Therefore, libraries based upon compact scaffolds that can display many side chains in a stereocontrolled fashion and have an architecture that mimics bio-molecules, such as peptides, are likely to be rich sources of drug leads.

Tetrahydro-pyrrolo[1,2-a]imidazole-2,5-diones provide a compact bicyclic scaffold for the preparation of large combinatorial libraries with the potential of up to five diversity side chains, four of which further diversify via chirality. In spite of this potential, the synthesis of this scaffold, and the biological activity of its derivatives, has received little attention relative to other scaffolds with large library potential. The tetrahydro-pyrrolo[1,2-a]imidazole-2,5-dione derivatives were shown to have cognition enhancing activities (Pinza et al., J. Med. Chem. 36:4214 (1993)) and some have been used as nootropic agents (European Patent No. 335483 to Pinza et al.). Aryl fused derivatives, e.g., 1H-imidazo[2,1-a]isoindole-2,5(3H, 9bH)-diones, are herbicides (Los, Ger. Offen. 2 700 269 (1977); U.S. Pat. No. 4,041,045 to Los; U.S. Pat. No. 4,090,860 to Ashkar; U.S. Pat. No. 4,067,718 to Ashkar), plant growth regulators (U.S. Pat. No. 4,093,441 to Ashkar), or have potential utility in treating diabetes and obesity (Tomoharu et al, Jpn Kokai Tokkyo Koho 124 pp. (2002); PCT International Publication No. WO 01/14386 to Toshio et al.). A solution phase synthesis of the aryl fused derivatives has been reported (Katritzky et al., J. Chem. Soc., Perkin Trans. 1: 1767 (2001)), but the literature synthesis of the aliphatic derivatives is not readily adaptable to the preparation of large combinatorial libraries and does not provide access to the 6-amino side chain diversity site.

The present invention is directed to overcoming these deficiencies in the art.

### SUMMARY OF THE INVENTION

The present invention relates to a combinatorial library comprising two or more tetrahydro-pyrrolo[1,2-a]imidazole-2,5-dione derivative compounds of the following formula: 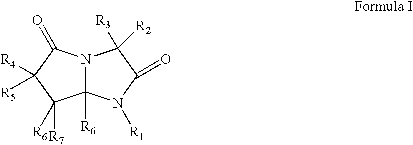
where:
R₁-R₈ are individually selected from the group consisting of: H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein any of R₁-R₈ can be further substituted with one or more halogen atoms or functional groups selected from the group consisting of: amides, ureas, ketones, sulfonamides, phosphoramides, alcohols, thiols, esters, amines, amidines, guanidines, carboxylic acids, sulfonic acids, phosphonic acids, and boronic acids;
R₄-R₇ can optionally be side chains with N, S, O, P, or B directly attached to ring of Formula I, substituted with any combination of H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein the directly attached N, S, or O can also be further attached to a carbonyl, sulfonyl, sulfinyl, or phosphoryl group to form a functional group selected from the group consisting of urea, amide, urethane, sulfonamides sulfinamide, sulfonylurea, sulfinylurea, and phosphoramide, wherein the functional group can be further substituted with H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl, wherein all of the side chains with N, S, O, P, or B can optionally contain one or more halogen atoms and a functional group selected from the group consisting of amide, urea, ketone, sulfonamide, phosphoramide, alcohol, thiol, ester, amine, amidine, guanidine, carboxylic acid, sulfonic acid, phosphonic acid, and boronic acid;
any combination of two substituents selected from R₁-R₈ can be covalently attached to each other to form another ring;
where the library comprises enantiomers, diastereomers, or a mixture of the enantiomers and the diastereomers of the compound;
with the proviso that the following conditions cannot all be true at the same time:
R₄═H, R₅═H, R₆═H, and R₇═H.

Another aspect of the present invention relates to a combinatorial library comprising two or more hexahydro-pyrrolo[1,2-a]pyrimnidine-2,6-dione derivative compounds of the following formula: 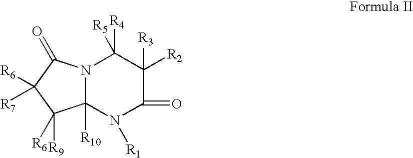
where:
R₁-R₁₀ are individually selected from the group consisting of: H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, alkylheteroaryl, and substituted alkylheteroaryl, wherein any of R₁-R₁₀ can be further substituted with one or more halogen atoms or functional groups selected from the group consisting of amides, ureas, ketones, sulfonamides, phosphoramides, alcohols, thiols, esters, amines, amidines, guanidines, carboxylic acids, sulfonic acids, phosphonic acids, and boronic acids;
R₆-R₉ can optionally be side chains with N, S, O, P, or B directly attached to ring of Formula I, substituted with any combination of H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein the directly attached N, S, or O can also be further attached to a carbonyl, sulfonyl, sulfinyl, or phosphoryl group to form a functional group selected from the group consisting of urea, amide, urethane, sulfonamides sulfmamide, sulfonylurea, sulfinylurea, and phosphoramide, wherein the functional group can be further substituted with H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl, wherein all of the side chains with N, S, O, P, or B can optionally contain one or more halogen atoms and a functional group selected from the group consisting of amide, urea, ketone, sulfonamide, phosphoramide, alcohol, thiol, ester, amine, amidine, guanidine, carboxylic acid, sulfonic acid, phosphonic acid, and boronic acid;
   any combination of two substituents selected from R₁-R₁₀ can be covalently attached to each other to form another ring;
   where the library comprises enantiomers, diastereomers, or a mixture of the enantiomers and the diastereomers of the compound.

The present invention also relates to a tetrahydro-pyrrolo[1,2-a]imidazole-2,5-dione derivative compound of the following formula: 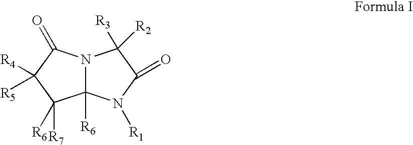
where:
R₁-R₈ are individually selected from the group consisting of: H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein any of R₁-R₈ can be further substituted with one or more halogen atoms or functional groups selected from the group consisting of: amides, ureas, ketones, sulfonamides, phosphoramides, alcohols, thiols, esters, amines, amidines, guanidines, carboxylic acids, sulfonic acids, phosphonic acids, and boronic acids;
R₄-R₇ can optionally be side chains with N, S, O, P, or B directly attached to ring of Formula I, substituted with any combination of H, C₁-C₁₂ alkyl heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein the directly attached N, S, or O can also be further attached to a carbonyl, sulfonyl, sulfinyl, or phosphoryl group to form a functional group selected from the group consisting of urea, amide, urethane, sulfonamides sulfinamide, sulfonylurea, sulfinylurea, and phosphoramide, wherein the functional group can be further substituted with H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl, wherein all of the side chains with N, S, O, P, or B can optionally contain one or more halogen atoms and a functional group selected from the group consisting of amide, urea, ketone, sulfonamide, phosphoramide, alcohol, thiol, ester, amine, amidine, guanidine, carboxylic acid, sulfonic acid, phosphonic acid, and boronic acid;
   any combination of two substituents selected from R₁-R₈ can be covalently attached to each other to form another ring;
   where the compound comprises an enantiomer, a diastereomer, or a mixture of the enantiomer and the diastereomer, or a pharmaceutically acceptable salt thereof;
   with the proviso that any of the following conditions cannot all be true at the same time:
R₄═H, R₅═H, R₆═H, and R₇═H;
R₁═R₃═R₅═R₆═R₇═R₈=H, R₂=isobutyl, and R₄=BocNH;
R₁═R₂═R₅═R₆═R₇═R₈=H, R₃=isobutyl, and R₄=BocNH;
R₁═R₃═R₄═R₆═R₇═R₈=H, R₂=isobutyl, and R₅=BocNH; or
R₁═R₂═R₄═R₆═R₇═R₈=H, R₃=isobutyl, and R₅=BocNH.

Another aspect of the present invention relates to a hexahydro-pyrrolo[1,2-a]pyrimidine-2,6-dione derivative compound of the following formula: 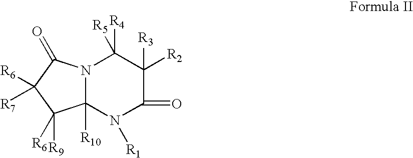
where:
R₁-R₁₀ are individually selected from the group consisting of: H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, alkylheteroaryl, and substituted alkylheteroaryl, wherein any of R₁-R₁₀ can be further substituted with one or more halogen atoms or functional groups selected from the group consisting of: amides, ureas, ketones, sulfonamides, phosphoramides, alcohols, thiols, esters, amines, amidines, guanidines, carboxylic acids, sulfonic acids, phosphonic acids, and boronic acids;
R₆-R₉ can optionally be side chains with N, S, O, P, or B directly attached to ring of Formula I, substituted with any combination of H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein the directly attached N, S, or O can also be further attached to a carbonyl, sulfonyl, sulfinyl, or phosphoryl group to form a functional group selected from the group consisting of urea, amide, urethane, sulfonamides sulfinamide, sulfonylurea, sulfinylurea, and phosphoramide, wherein the functional group can be further substituted with H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl, wherein all of the side chains with N, S, O, P, or B can optionally contain one or more halogen atoms and a functional group selected from the group consisting of amide, urea, ketone, sulfonamide, phosphoramide, alcohol, thiol, ester, amine, amidine, guanidine, carboxylic acid, sulfonic acid, phosphonic acid, and boronic acid;
   any combination of two substituents selected from R₁-R₁₀ can be covalently attached to each other to form another ring;
   wherein the compound comprises an enantiomer, a diastereomer, a mixture of the enantiomer and the diastereomer, or a pharmaceutically acceptable salt thereof.

The present invention also relates to a method of preparing a tetrahydro-pyrrolo[1,2-a]imidazole-2,5-dione derivative compound. The method first involves providing a functionalized resin solid support. Next, the functionalized resin solid support is reacted with amino acids under conditions effective to produce a resin bound dipeptide or tripeptide alcohol intermediate compound. Then, the resin bound dipeptide or tripeptide alcohol intermediate compound is oxidized under conditions effective to convert the resin bound dipeptide or tripeptide alcohol intermediate compound to a resin bound dipeptide or tripeptide aldehyde intermediate compound. Finally, the resin bound dipeptide or tripeptide aldehyde intermediate compound is cyclized under conditions effective to produce the tetrahydro-pyrrolo[1,2-a]imidazole-2,5-dione derivative compound.

Another aspect of the present invention relates to a method of preparing a hexahydro-pyrrolo[1,2-a]pyrimidine-2,6-dione derivative compound. The method first involves providing a functionalized resin solid support. Next, the functionalized resin solid support is reacted with amino acids under conditions effective to produce a resin bound dipeptide or tripeptide alcohol intermediate compound. Then, the resin bound dipeptide or tripeptide alcohol intermediate compound is oxidized under conditions effective to convert the resin bound dipeptide or tripeptide alcohol intermediate compound to a resin bound dipeptide or tripeptide aldehyde intermediate compound. Finally, the resin bound dipeptide or tripeptide aldehyde intermediate compound is cyclized under conditions effective to produce the hexahydro-pyrrolo[1,2-a]pyrimidine-2,6-dione derivative compound.

Yet another aspect of the present invention relates to a method of preparing a tetrahydro-pyrrolo[1,2-a]imidazole-2,5-dione derivative compound. The method first involves treating amino acids under conditions effective to produce a dipeptide or tripeptide alcohol intermediate compound. Then, the dipeptide or tripeptide alcohol intermediate compound is oxidized under conditions effective to convert the dipeptide or tripeptide alcohol intermediate compound to a dipeptide or tripeptide aldehyde intermediate compound. Finally, the dipeptide or tripeptide aldehyde intermediate compound is cyclized under alcohol refluxing conditions to produce the tetrahydro-pyrrolo[1,2-a]imidazole-2,5-dione derivative compound, where the above steps of treating, oxidizing, and cyclizing are all performed in solution.

Another aspect of the present invention relates to a method of preparing a hexahydro-pyrrolo[1,2-a]pyrimidine-2,6-dione derivative compound. The method first involves treating amino acids under conditions effective to produce a dipeptide or tripeptide alcohol intermediate compound. Then, the dipeptide or tripeptide alcohol intermediate compound is oxidized under conditions effective to convert the dipeptide or tripeptide alcohol intermediate compound to a dipeptide or tripeptide aldehyde intermediate compound. Finally, the dipeptide or tripeptide aldehyde intermediate compound is cyclized under alcohol refluxing conditions to produce the hexahydro-pyrrolo[1,2-a]pyrimidine-2,6-dione derivative compound, where the above steps of treating, oxidizing, and cyclizing are all performed in solution.

The present invention provides novel and efficient methods for synthesizing compact, bicyclic scaffolds that are adapatable to the preparation of large combinatorial libraries. The disclosed bicyclic scaffolds are particularly advantageous because they are compact and allow numerous side chains to be attached in a stereocontrolled fashion. The number and stereochemistry of these side chains provide the potential to synthesize extremely large libraries. The compact structure of the scaffolds and their similarity to certain peptide secondary structures, such as beta-turns, make it likely that numerous members of the library will bind to drug targets (i.e., high “hit rate”) when the disclosed libraries are screened against appropriate drug targets.

### DETAILED DESCRIPTION OF THE INVENTION

The present invention relates to a combinatorial library comprising two or more tetrahydro-pyrrolo[1,2-a]imidazole-2,5-dione derivative compounds of the following formula: 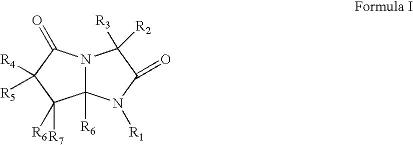
where:
R₁-R₈ are individually selected from the group consisting of: H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein any of R₁-R₈ can be further substituted with one or more halogen atoms or functional groups selected from the group consisting of: amides, ureas, ketones, sulfonamides, phosphoramides, alcohols, thiols, esters, amines, amidines, guanidines, carboxylic acids, sulfonic acids, phosphonic acids, and boronic acids;
R₄-R₇ can optionally be side chains with N, S, O, P, or B directly attached to ring of Formula I, substituted with any combination of H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein the directly attached N, S, or O can also be further attached to a carbonyl, sulfonyl, sulfinyl, or phosphoryl group to form a functional group selected from the group consisting of urea, amide, urethane, sulfonamides sulfinamide, sulfonylurea, sulfinylurea, and phosphoramide, wherein the functional group can be further substituted with H, C₁- C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl, wherein all of the side chains with N, S, O, P, or B can optionally contain one or more halogen atoms and a functional group selected from the group consisting of amide, urea, ketone, sulfonamide, phosphoramide, alcohol, thiol, ester, amine, amidine, guanidine, carboxylic acid, sulfonic acid, phosphonic acid, and boronic acid;
   any combination of two substituents selected from R₁-R₈ can be covalently attached to each other to form another ring;
   where the library comprises enantiomers, diastereomers, or a mixture of the enantiomers and the diastereomers of the compound;
   with the proviso that the following conditions cannot all be true at the same time:
R₄═H, R₅═H, R₆═H, and R₇═H.

As used herein, the term alkyl refers to a saturated aliphatic hydrocarbon including straight chain and branched chain groups. The alkyl group may be substituted or unsubstituted. Suitable substituents include, but are not limited to, cycloalkyl, aryl, heteroaryl, heteroalicyclic, hydroxy, alkoxy, aryloxy, thiohydroxy, thioalkoxy, thioaryloxy, cyano, halogen, carbonyl, thiocarbonyl, carboxy, nitro, silyl, and amino. A cyclic alkyl group refers to an all carbon monocyclic or fused ring (i.e., rings which share an adjacent pair of atoms) group wherein one or more of the rings does not have a completely conjugated pi-electron system. Suitable cyclic alkyl groups include, but are not limited to, cyclopropane, cyclobutane, cyclopentane, cyclopentene, cyclohexane, cyclohexadiene, cycloheptane, and cycloheptatriene. A cyclic alkyl group may be substituted or unsubstituted. Suitable substituents include those described above for alkyl groups.

As used herein, an aryl group refers to an all carbon monocyclic or fused-ring polycyclic group having a completely conjugated pi-electron system. Suitable examples of aryl groups include, but are not limited to, phenyl, benzyl, benzoyl, naphthalenyl, and anthracenyl. The aryl group may be substituted or unsubstituted. Suitable substituents include, but are not limited to, alkyl, hydroxy, alkoxy, aryloxy, thiohydroxy, thioalkoxy, thioaryloxy, cyano, nitro, carbonyl, thiocarbonyl, carboxy, sulfinyl, sulfonyl, amino, halogen, and triohalomethyl.

As used herein, a heteroaryl group refers to a monocyclic or fused ring group having in the ring(s) one or more heteroatoms, such as sulfur, nitrogen, and oxygen, and in addition having a completely conjugated pi-electron system. Suitable heteroaryl groups include, but are not limited to, pyrrole, furan, thiophene, imidazole, oxazole, thiazole, pyrazole, pyridine, pynmidine, quinoline, isoquinoline, purine, and carbazole. The heteroaryl group may be substituted or unsubstituted. Suitable substituents include, but are not limited to, alkyl, cycloalkyl, hydroxy, alkoxy, aryloxy, thiohydroxy, thioalkoxy, thioaryloxy, cyano, nitro, carbonyl, thiocarbonyl, carboxy, sulfinyl, sulfonyl, amino, halogen, and triohalomethyl.

As used herein, a “combinatorial library” is an intentionally created collection of differing molecules which can be created by the techniques set forth below or otherwise and screened for activity in a variety of formats (e.g., libraries of soluble molecules or libraries of compounds attached to a solid support). A “combinatorial library” involves successive and/or parallel rounds of chemical synthesis based on a common starting structure. The combinatorial libraries can be screened in a variety of assays useful for assessing biological or chemical activities. Compounds disclosed in the prior art that are not in an intentionally created collection are not part of a “combinatorial library” of the present invention. In addition, compounds that are part of an unintentional or undesired mixture are not part of a “combinatorial library” of the present invention.

In accordance with the present invention, the combinatorial library synthesis can be carried out either manually or through the use of an automated process. For manual synthesis, the chemical manipulations would be performed by a scientist or technician. For automated synthesis, the chemical manipulations would typically be performed robotically. The choice and implementation of such techniques is within the skill of one of ordinary skill in the art of combinatorial chemistry.

In one embodiment of the present invention, one or more of R₄-R₇ are side chains with an attached where the attached N is a part of a functional group selected from the group consisting of amide, urea, urethane, amidine, guanidine, sulfonamide, sulfonylurea, phosphoramide, unsubstituted amine, mono-alkyl amine, aryl amine, heteroaryl amine, dialkyl amine, and diaryl amine, where one or both aryl groups can be a heteroaryl group, where the functional group can be further substituted with H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl aryl and substituted aryl, heteroaryl and substituted heteroaryl, alkylaryl and substituted alkylaryl, or alkylheteroaryl and substituted alkylheteroaryl.

In another embodiment of the present invention, the combinatorial library includes at least one compound having the following formula:

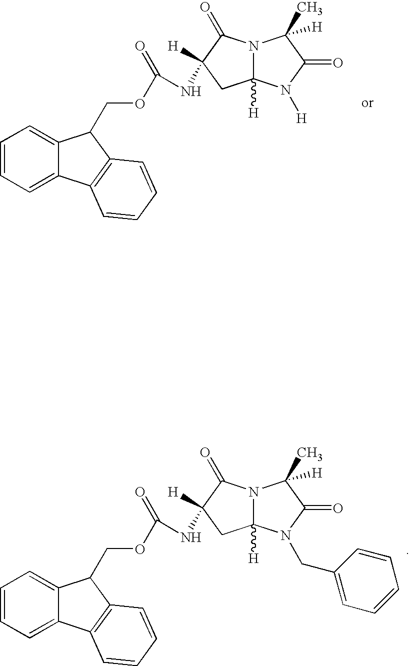

Another aspect of the present invention relates to a combinatorial library comprising two or more hexahydro-pyrrolo[1,2-a]pyrimidine-2,6-dione derivative compounds of the following formula: 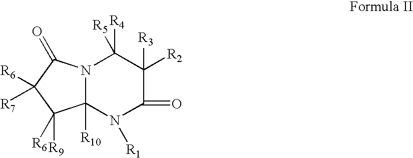
where:
R₁-R₁₀ are individually selected from the group consisting of: H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, alkylheteroaryl, and substituted alkylheteroaryl, wherein any of R₁-R₁₀ can be further substituted with one or more halogen atoms or functional groups selected from the group consisting of: amides, ureas, ketones, sulfonamides, phosphoramides, alcohols, thiols, esters, amines, amidines, guanidines, carboxylic acids, sulfonic acids, phosphonic acids, and boronic acids;
R₆-R₉ can optionally be side chains with N, S, O, P, or B directly attached to ring of Formula I, substituted with any combination of H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein the directly attached N, S, or O can also be further attached to a carbonyl, sulfonyl, sulfinyl, or phosphoryl group to form a functional group selected from the group consisting of urea, amide, urethane, sulfonamides sulfmamide, sulfonylurea, sulfinylurea, and phosphoramide, wherein the functional group can be further substituted with H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl heteroaryl and substituted heteroaryl, alkylaryl and substituted alkylaryl, or alkylheteroaryl and substituted alkylheteroaryl, wherein all of the side chains with N, S, O, P, or B can optionally contain one or more halogen atoms and a functional group selected from the group consisting of amide, urea, ketone, sulfonamide, phosphoramide, alcohol, thiol, ester, amine, amidine, guanidine, carboxylic acid, sulfonic acid, phosphonic acid, and boronic acid;
   any combination of two substituents selected from R₁-R₁₀ can be covalently attached to each other to form another ring;
   where the library comprises enantiomers, diastereomers, or a mixture of the enantiomers and the diastereomers of the compound.

In another embodiment of the present invention, one or more of R₆-R₉ are side chains with an attached where the attached N is a part of a functional group selected from the group consisting of amide, urea, urethane, amidine, guanidine, sulfonamide, sulfonylurea, phosphoramide, unsubstituted amine, mono-alkyl amine, aryl amine, heteroaryl amine, dialkyl amine, and diaryl amine, where one or both aryl groups can be a heteroaryl group, where the functional group can be further substituted with H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl.

In another embodiment of the present invention, the combinatorial library includes at least one compound having the above Formula (II), where
R₁═R₂═R₃═R₄═R₅═R₇═R₈═R₉═R₁₀=H and R₆=FmocNH.

The present invention also relates to a tetrahydro-pyrrolo[1,2-a]iimidazole-2,5-dione derivative compound of the following formula: 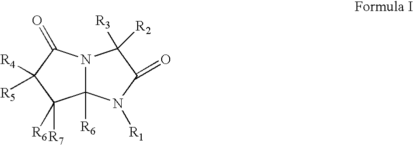
where:
R₁-R₈ are individually selected from the group consisting of: H, C₁-C₁₂ alkyl, heteroatom and multiple heteroatom substituted C₁-C₁₂ alkyl C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein any of R₁-R₈ can be further substituted with one or more halogen atoms or functional groups selected from the group consisting of: amides, ureas, ketones, sulfonamides, phosphoramides, alcohols, thiols, esters, amines, amidines, guanidines, carboxylic acids, sulfonic acids, phosphonic acids, and boronic acids;
R₄-R₇ can optionally be side chains with N, S, O, P, or B directly attached to ring of Formula I, substituted with any combination of H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein the directly attached N, S, or O can also be further attached to a carbonyl, sulfonyl, sulfinyl, or phosphoryl group to form a functional group selected from the group consisting of urea, amide, urethane, sulfonamides sulfmamide, sulfonylurea, sulfinylurea, and phosphoramide, wherein the functional group can be further substituted with H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl, wherein all of the side chains with N, S, O, P, or B can optionally contain one or more halogen atoms and a functional group selected from the group consisting of amide, urea, ketone, sulfonamide, phosphoramide, alcohol, thiol, ester, amine, amidine, guanidine, carboxylic acid, sulfonic acid, phosphonic acid, and boronic acid;
   any combination of two substituents selected from R₁-R₈ can be covalently attached to each other to form another ring;
   where the compound comprises an enantiomer, a diastereomer, or a mixture of the enantiomer and the diastereomer, or a pharmaceutically acceptable salt thereof,
   with the proviso that any of the following conditions cannot all be true at the same time:
R₄═H, R₅═H, R₆═H, and R₇═H;
R₁═R₃═R₅═R₆═R₇═R₈=H, R₂=isobutyl, and R₄=BocNH;
R₁═R₂═R₅═R₆═R₇═R₈=H, R₃=isobutyl, and R₄=BocNH;
R₁═R₃═R₄═R₆═R₇═R₈=H, R₂=isobutyl, and R₅=BocNH; or
R₁═R₂═R₄═R₆═R₇═R₉=H, R₃=isobutyl, and R₅=BocNH.

Another aspect of the present invention relates to a hexahydro-pyrrolo[1,2-a]pyrimidine-2,6-dione derivative compound of the following formula: 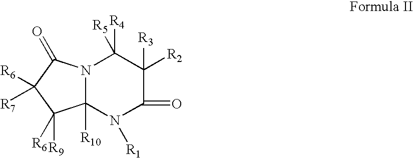
where:
R₁-R₁₀ are individually selected from the group consisting of: H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, alkylheteroaryl, and substituted alkylheteroaryl, wherein any of R₁-R₁₀ can be further substituted with one or more halogen atoms or functional groups selected from the group consisting of: amides, ureas, ketones, sulfonamides, phosphoramides, alcohols, thiols, esters, amines, amidines, guanidines, carboxylic acids, sulfonic acids, phosphonic acids, and boronic acids;
R₆-R₉ can optionally be side chains with N, S, O, P, or B directly attached to ring of Formula I, substituted with any combination of H, C₁-C₁₂ alkyl heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl alkylaryl and substituted alkylaryl, and alkylheteroaryl and substituted alkylheteroaryl, where the directly attached N, S, or O can also be further attached to a carbonyl, sulfonyl, sulfinyl, or phosphoryl group to form a functional group selected from the group consisting of urea, amide, urethane, sulfonamides sulfmamide, sulfonylurea, sulfinylurea, and phosphoramide, where the functional group can be further substituted with H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl, where all of the side chains with N, S, O, P, or B can optionally contain one or more halogen atoms and a functional group selected from the group consisting of amide, urea, ketone, sulfonamide, phosphoramide, alcohol, thiol, ester, amine, amidine, guanidine, carboxylic acid, sulfonic acid, phosphonic acid, and boronic acid;
   any combination of two substituents selected from R₁-R₁₀ can be covalently attached to each other to form another ring;
   wherein the compound comprises an enantiomer, a diastereomer, or a mixture of the enantiomer and the diastereomer, or a pharmaceutically acceptable salt thereof

In addition, the present invention relates to a method of preparing a hexahydro-pyrrolo[1,2-a]pyrimidine-2,6-dione derivative compound. The method first involves providing a functionalized resin solid support. Next, the functionalized resin solid support is reacted with amino acids under conditions effective to produce a resin bound dipeptide or tripeptide alcohol intermediate compound. Then, the resin bound dipeptide or tripeptide alcohol intermediate compound is oxidized under conditions effective to convert the resin bound dipeptide or tripeptide alcohol intermediate compound to a resin bound dipeptide or tripeptide aldehyde intermediate compound. Finally, the resin bound dipeptide or tripeptide aldehyde intermediate compound is cyclized under conditions effective to produce the hexahydro-pyrrolo[1,2-a]pyrimidine-2,6-dione derivative compound.

In one embodiment of the method, the tetrahydro-pyrrolo[1,2-a]imidazole-2,5-dione derivative compound has the following formula: 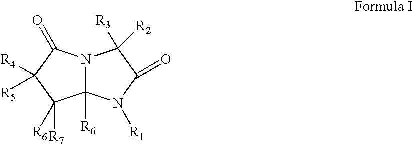
where:
R₁-R₈ are individually selected from the group consisting of: H, C₁-C₁₂ alkyl heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein any of R₁-R₈ can be further substituted with one or more halogen atoms or functional groups selected from the group consisting of: amides, ureas, ketones, sulfonamides, phosphoramides, alcohols, thiols, esters, amines, amidines, guanidines, carboxylic acids, sulfonic acids, phosphonic acids, and boronic acids;
R₄-R₇ can optionally be side chains with N, S, O, P, or B directly attached to ring of Formula I, substituted with any combination of H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, where the directly attached N, S, or O can also be further attached to a carbonyl, sulfonyl, sulfinyl, or phosphoryl group to form a functional group selected from the group consisting of urea, amide, urethane, sulfonamides sulfinamide, sulfonylurea, sulfinylurea, and phosphoramide, where the functional group can be further substituted with H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl, where all of the side chains with N, S, O, P, or B can optionally contain one or more halogen atoms and a functional group selected from the group consisting of amide, urea, ketone, sulfonamide, phosphoramide, alcohol, thiol, ester, amine, amidine, guanidine, carboxylic acid, sulfonic acid, phosphonic acid, and boronic acid;
   any combination of two substituents selected from R₁-R₈ can be covalently attached to each other to form another ring;
   where the compound comprises an enantiomer, a diastereomer, or a mixture of the enantiomer and the diastereomer, or a pharmaceutically acceptable salt thereof

A general synthetic reaction scheme for the preparation of tetrahydro-pyrrolo[1,2-a]imidazole-2,5-dione derivative compound in accordance with the present invention is illustrated in Scheme 1 below. It should be appreciated that the chemical compounds described herein are merely exemplary and other groups can be used.

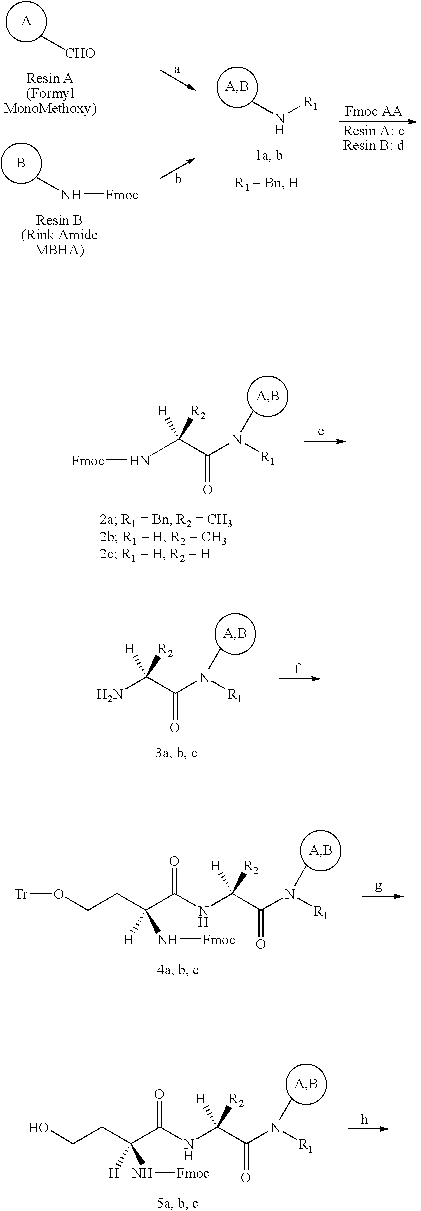

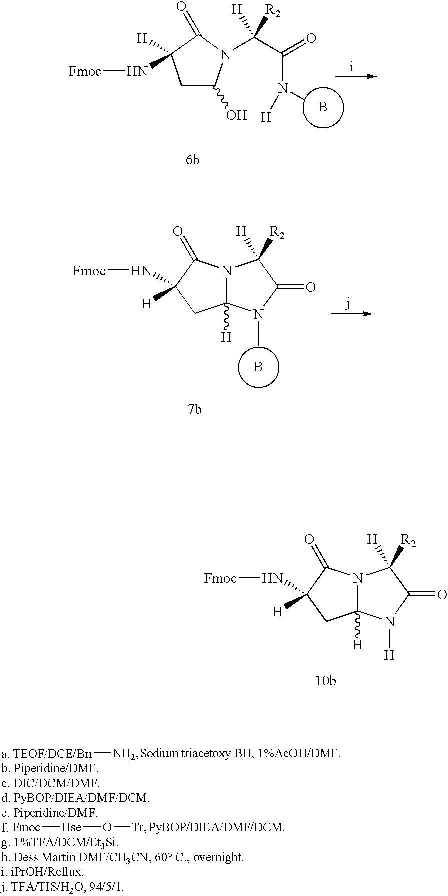

In order to develop a synthesis of the bicyclic scaffold compounds of the present invention that is adaptable to the preparation of large combinatorial libraries, it is preferable to carry out as much of the synthesis on a solid support as possible. Solid supported syntheses remove the need for workups and purification of intermediates, allow the use of efficient “mix and split” large library synthesis strategies, and thus are particularly advantageous for multistep reaction sequences. As used herein, a solid support is an insoluble substrate that has been appropriately derivatized such that a chemical molecule can be attached to the surface of the substrate through standard chemical methods. Suitable solid supports include, but are not limited to, beads and particles. The solid support can include many different materials which are capable of being functionalized through synthetic methods or already include a suitable functional group. Examples of such materials include, but are not limited to, polymers, plastics, resins, polysaccharides, silicon or silica based materials, carbon, metals, inorganic glasses, and membranes.

As described above, the solid support can be provided with a suitable functionality already present or the solid support can be chemically modified such that a desired chemical molecule is attached to the support surface. The choice of functionality used for attaching a chemical molecule to the solid support will depend on the nature of the molecule to be attached and the type of solid support.

In one embodiment of the present invention, the functionalized resin solid support is an amine resin. The amine resin can be prepared by treating a Formyl Monomethoxy (FMP) resin (Sarantakis et al., Tetrahedron Lett. 38:7325 (1997); Fivush et al., Tetrahedron Lett., 38:7151 (1997), which are hereby incorporated by reference in their entirety) or a Rink Amide (MBHA) resin (Novabiochem Catalog 2:25 (2002/2003), which is hereby incorporated by reference in its entirety), depending upon whether N−1 is to be substituted or unsubstituted, respectively.

The amine resin substituent can be introduced by reductive amination of the aldehyde moiety in the FMP solid support (Resin A in Scheme 1). This reaction is illustrated with benzyl amine and NaBH(OAc)₃ in N,N-dimethylforamide (DMF) containing 1% HOAc to give the tethered benzyl amine 1a (Makara et al., Tetrahedron Lett., 42:4123 (2002), which is hereby incorporated by reference in its entirety). Removal of the Fmoc protecting group from the MBHA solid support (Resin B in Scheme 1) produces the unsubstituted amine resin 1b (Fantenot et al., Pept. Res. 4:19 (1991), which is hereby incorporated by reference in its entirety).

Acylation of the resulting amines 1a,b (i.e., the functionalized resin solid support) with a first amino acid, such as a Fmoc-L-amino acid, provides tethered amides, as illustrated with Fmoc-L-Ala and Fmoc-L-Gly 2a,b,c (i.e., the resin bound amino acid intermediate compound). In another embodiment, the first amino acid is Fmoc-D-amino acid. This acylation step may require considerable optimization on resins A & B, where it can be carried out by using a coupling reagent selected from O-(7-azabenzotriazol-1-yl)-1,1,3,3-tetramethyluronium hexafluorophosphate (HATU), O-(benzotriazol-1-yl)-1,1,3,3-tetramethyluronium hexafluorophosphate (HBTU), O-(benzotriazol-1-yl)-1,1,3,3-tetramethyluronium tetrafluoroborate (TBTU), 1,3-diisopropylcarbodiimide (DIC), 1-(3-dimethylaminopropyl)-3-ethylcarbodiimide hydrochloride (EDCI), 1-benzotriazolyloxytris(pyrrolidino)phosphonium hexafluorophosphate (PyBOP) and bromotris(pyrrolidino)phosphonium hexafluorophosphate (PyBroP). Preferably, the coupling reagent is DIC for this first coupling step with resin A, while the coupling reagent is PyBOP for resin B. Removal of Fmoc protecting group from 2a,b,c can be effected with a 25% piperidine in DMF for about 15 minutes, followed by coupling of protected homoserine (Fmoc-L-Hse-O-Tr) to afford the trityl dipeptide ethers 4a,b,c. Preferably, the coupling reagent is PyBOP on both resins for this second coupling step. To ensure that complete acylation occurs the resins can be subjected to these coupling conditions more than once.

Removal of the trityl group can be performed using 1% trifluoroacetic acid (TFA) in dichloromethane (DCM) and silanes to scavenge the trityl cations (Barlos et al., Tetrahedron Lett. 32 (4):471 (1991), which is hereby incorporated by reference in its entirety), providing the resin tethered dipeptide alcohols 5a,b,c (i.e., the resin bound dipeptide alcohol intermediate compound). In another embodiment, this step can produce a resin bound tripeptide alcohol intermediate compound. A number of oxidation reagents, such as Swern, pyridine-SO₃, tetra-n-propylammonium perruthenate(VII), pyridinium chlorochromate, pyridinium dichromate, and Dess-Martin periodinane, can be used to convert the alcohol side chain in 5b, while tethered to the resin, into the corresponding aldehyde (i.e., the resin bound dipeptide aldehyde intermediate compound) needed to trigger the cyclization reaction to 5-hydroxy-pyrrolidin-2-one 6b. In another embodiment, the aldehyde can be a resin bound tripeptide aldehyde intermediate compound. Various conditions, such as Ac₂O, Toluene/p-toluene sulfonic acid (TsOH), and refluxing in isopropanol for 3 days, can be used to complete the cyclization to the final bicyclic scaffold (7b) while still tethered to the resin. The most preferable reagents for the conversion of alcohol 5b to bicyclic compound 7b is Dess-Martin periodinane in CH₃CN/DMF (2/1) at 60° C. overnight, followed by refluxing in alcohol, such as isopropanol, for 4 days.

In another embodiment of the present invention, the resin bound dipeptide alcohol intermediate (i.e., the resin tethered dipeptide alcohols 5a,b,c in Scheme 1) can be cleaved from the solid support and the oxidation and cyclization steps can be performed in solution. The resin bound dipeptide alcohol intermediate can be cleaved from the solid support by using TFA/DCM/triethyl silane or TFA/triisopropylsilane (TIS)/H₂O. The reaction scheme for these embodiments of the present invention is illustrated in Scheme 2 below.

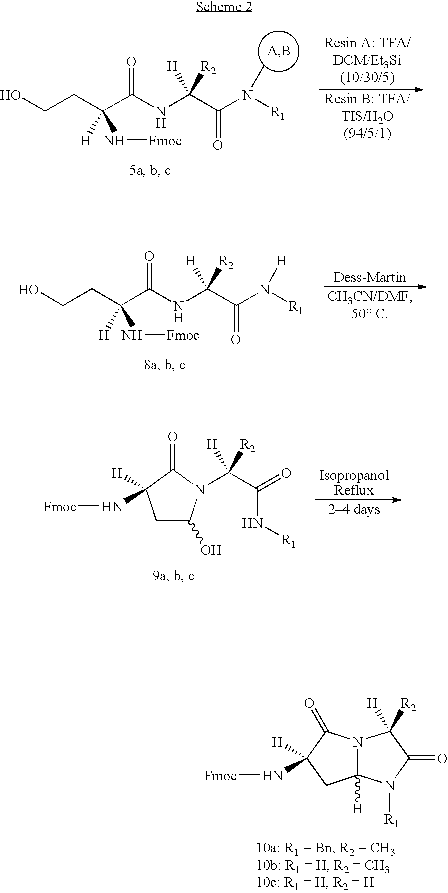

Another aspect of the present invention relates to a method of preparing a hexahydro-pyrrolo[1,2-a]pyrimidine-2,6-dione derivative compound. The method first involves providing a functionalized resin solid support. Next, the functionalized resin solid support is reacted with amino acids under conditions effective to produce a resin bound dipeptide or tripeptide alcohol intermediate compound. Then, the resin bound dipeptide or tripeptide alcohol intermediate compound is oxidized under conditions effective to convert the resin bound dipeptide or tripeptide alcohol intermediate compound to a resin bound dipeptide or tripeptide aldehyde intermediate compound. Finally, the resin bound dipeptide or tripeptide aldehyde intermediate compound is cyclized under conditions effective to produce the hexahydro-pyrrolo[1,2-a]pyrimidine-2,6-dione derivative compound.

In another embodiment of the present invention, the hexahydro-pyrrolo[1,2-a]pyrimidine-2,6-dione derivative compound has the following formula: 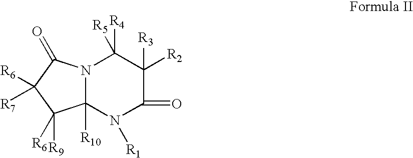
where:
R₁-R₁₀ are individually selected from the group consisting of: H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, alkylheteroaryl, and substituted alkylheteroaryl, where any of R₁-R₁₀ can be further substituted with one or more halogen atoms or functional groups selected from the group consisting of: amides, ureas, ketones, sulfonamides, phosphoramides, alcohols, thiols, esters, amines, amidines, guanidines, carboxylic acids, sulfonic acids, phosphonic acids, and boronic acids;
R₆-R₉ can optionally be side chains with N, S, O, P, or B directly attached to ring of Formula I, substituted with any combination of H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, where the directly attached N, S, or O can also be further attached to a carbonyl, sulfonyl, sulfinyl, or phosphoryl group to form a functional group selected from the group consisting of urea, amide, urethane, sulfonamides suffmamide, sulfonylurea, sulfinylurea, and phosphoramide, where the functional group can be further substituted with H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl, where all of the side chains with N, S, O, P, or B can optionally contain one or more halogen atoms and a functional group selected from the group consisting of amide, urea, ketone, sulfonamide, phosphoramide, alcohol, thiol, ester, amine, amidine, guanidine, carboxylic acid, sulfonic acid, phosphonic acid, and boronic acid;
   any combination of two substituents selected from R₁-R₁₀ can be covalently attached to each other to form another ring;
   wherein the compound comprises an enantiomer, a diastereomer, or a mixture of the enantiomer and the diastereomer, or a pharmaceutically acceptable salt thereof.

In another embodiment of the present invention, the step of reacting the functionalized resin solid support involves a first coupling step where a first amino acid is coupled to the functionalized resin solid support under conditions effective to produce a resin bound amino acid intermediate compound and a second coupling step where a second amino acid is coupled to the resin bound amino acid intermediate compound under conditions effective to produce a resin bound dipeptide or tripeptide alcohol intermediate compound. In another embodiment, the first amino acid can be a Fmoc-β-alanine.

Yet another aspect of the present invention relates to a method of preparing a tetrahydro-pyrrolo[1,2-a]imidazole-2,5-dione derivative compound. The method first involves treating amino acids under conditions effective to produce a dipeptide or tripeptide alcohol intermediate compound. Then, the dipeptide or tripeptide alcohol intermediate compound is oxidized under conditions effective to convert the dipeptide or tripeptide alcohol intermediate compound to a dipeptide or tripeptide aldehyde intermediate compound. Finally, the dipeptide or tripeptide aldehyde intermediate compound is cyclized under alcohol refluxing conditions to produce the tetrahydro-pyrrolo[1,2-a]imidazole-2,5-dione derivative compound, where the above steps of treating, oxidizing, and cyclizing are all performed in solution. In another embodiment, the oxidizing step is carried out with Dess-Martin periodinane.

Another aspect of the present invention relates to a method of preparing a hexahydro-pyrrolo[1,2-a]pyrimidine-2,6-dione derivative compound. The method first involves treating amino acids under conditions effective to produce a dipeptide or tripeptide alcohol intermediate compound. Then, the dipeptide or tripeptide alcohol intermediate compound is oxidized under conditions effective to convert the dipeptide or tripeptide alcohol intermediate compound to a dipeptide or tripeptide aldehyde intermediate compound. Finally, the dipeptide or tripeptide aldehyde intermediate compound is cyclized under alcohol refluxing conditions to produce the hexahydro-pyrrolo[1,2-a]pyrimidine-2,6-dione derivative compound, where the above steps of treating, oxidizing, and cyclizing are all performed in solution.

The combinatorial libraries of the present invention can be screened against various drug targets that bind to peptide or proteins, such as enzymes, receptors, and transcription factor complexes, to identify appropriate drug compounds.

### EXAMPLES

The following examples are provided to illustrate embodiments of the present invention but are by no means intended to limit its scope.

### Example 1

### General Methods and Materials for the Novel Synthesis of Tetrahydro-pyrrolo[1,2-a]imidazole-2,5-dione Derivatives

All solution phase reactions were carried out under nitrogen atmosphere using dry glassware that had been oven dried. A Mettler Toledo Bohdan (now AutoChem, Millersville, Md.) MiniBlock™ parallel synthesizer fitted with 20 ml size glass reactor tubes (containing glass fritted filtering disks at the bottom) was used for all solid phase reactions and with the orbital shaker speed set at 800 rpm and under a nitrogen atmosphere (using the MiniBlock gas manifold) to stir the reactions. Since resins can occasionally be difficult to stir well with orbital stirring alone, a slow stream of N₂ was also bubbled through the reaction mixture when needed by immersing a plastic pipet into the reaction mixture with the MiniBlock gas manifold removed. In these cases, solvent evaporation is accelerated (especially DCM) and the most volatile solvent needs to be added periodically to replace the reaction solvent volume lost due to evaporation. The ability to stir the resin well with the Bohdan orbital shaker was also affected by the size of the reaction tubes (e.g. 20 mL tubes worked better than 5 mL tubes) and by the density of the reaction solvent mixture. The best mixing results were typically obtained when the density of the solvent mixture was roughly the same as the density of the resin, because the resin then remained suspended and dispersed in the reaction mixture rather than clumping at the bottom of the tube or floating on the top. All solvents were purchased from Aldrich (Milwaukee, Wis.) or Acros (Pittsburgh, Pa.) and used without further purification. Dess-Martin periodinane, diisopropylethylamine (DIEA), DIC, TIS, triethyl silane (Et₃SiH), and 1-hydroxybenzotriazole (HOBT) were purchased from Acros. All amino acids (L-amino acids), PyBOP, other coupling reagents and the Rink Amide MBHA resin were purchased from Novabiochem (La Jolla, Calif.). Formyl Monomethoxy resin (FMP) was purchased from Senn chemicals Inc (San Diego, Calif.). Solution phase reactions were monitored by thin-layer chromatography (TLC), using silica gel F (200 microns) coated on aluminium foil and purchased from Analtech Inc. (Newark, Del.). Visualization of TLC slides was accomplished with UV light or iodine staining. Flash Chromatography (herein meant to indicate column chromatography under pressure using various flow rates) was performed with the indicated solvents using silica gel 60 (particle size 0.040-0.063 mm) purchased from E. Merck (Gibbstown, N.J.). Yields refer to chromatographically and spectroscopically pure compounds unless otherwise stated. ¹H NMR spectra were recorded on a Varian (Palo Alto, Calif.) VXR-400S 400 MHz spectrometer at ambient temperature and all values are reported in parts per milion (ppm, δ) downfield from tetramethylsilane (TMS). Exchangeable protons were determined by adding a drop of D₂O to the NMR sample and retaking the NMR spectrum. Protons that disappear are assumed to have exchanged with deuterium and are NH or OH protons. High and low resolution mass spectra were recorded on a ThermoFinnigan (Waltham, Mass.) MAT 95 XL spectrometer fitted with a ESI II source. LC/MS experiments were carried out using a ThermoFinnigan LCQ Advantage Ion Trap instrument and were used to determine the ratio of individual diastereomers of the final bicyclic structures 10.

Resins are typically dried in vacuo for two hours at room temperature unless indicated otherwise. Excess solvents or solutions were removed from the resin by applying N₂ pressure to the top of the reactor tube with the exit port below the fritted disc open thereby filtering through the reactor fritted disc.

The success of the reactions in Scheme 1 was initially determined by cleaving the bicyclic compound 7b from the resin using TFA/TIS/H₂O (94/5/1) and analysis of the crude product 10b. Using the best reagents for the two cyclization steps, followed by cleavage from the resin, 10b was isolated in a 12% overall yield from MBHA resin. The use of DCE as the solvent for the oxidation reaction reduced the overall yield of 10b to the 5% range. Likewise, the use of toluene/TsOH to promote the final cyclization reduced the overall yield of 10b to the 2% range. The reduced yields of 10b with different oxidation reagents and solvents was primarily due to incomplete oxidation on the resin (alcohol 8b, Scheme 2, was recovered). The incomplete oxidation with other solvents might be attributed to formation of peptide aggregates within the gel of the solid support resulting in reduced access of the reagents to the alcohol moiety (Sheehan et al., J. Am. Chem. Soc. 77:1067 (1955), which is hereby incorporated by reference in its entirety). The driving force for this association may be the formation of hydrogen bonds which can be disrupted with polar solvents such as DMF (Beyermann et al., Int. J. Peptide Protein Res., 37 (4):252 (1991), which is hereby incorporated by reference in its entirety). The low yield in converting alcohol 5b to monocyclic intermediate 6b on the solid phase under the best conditions was confirmed by showing that the alcohol 5b can be cleaved from the resin to give an 82% yield of 8b (Scheme 2), whereas, after oxidation, cleavage of the monocyclic intermediate 6b from the resin gives only a 25% yield of 9b, both after purification.

Since incomplete oxidation of alcohols 5a,b,c led to reduced overall yields of the bicyclic product 10a,b,c, even when the oxidation step was optimized with polar solvent mixtures (CH₃CN/DMF), carrying out the oxidation and cyclization steps in solution was evaluated. The alcohols 5a,b,c were cleaved from the resin using TFA/DCM/Et₃Si (10/30/1 for 5a) or TFA/TIS/H₂O (94/5/1 for 5b,c) and 8a,b,c were obtained in 82, 82 and 84% overall yields (after purification), respectively, from the starting resins. Dess-Martin oxidation (Dess et al., J. Org. Chem. 48:4155 (1983), which is hereby incorporated by reference in its entirety) of 8a,b,c in CH₃CN containing a few drops of DMF Oust enough to fully dissolve the peptide) at 50° C. afforded the monocyclic intermediates 9a,b,c in yields over 80%. Completion of the cyclization in refluxing isopropanol afforded the bicyclic structures 10a,b,c in yields of 75, 68 and 58%, respectively.

These results showed that the synthesis of the peptide alcohols 5a,b,c occured in overall yields above 80% with 5 steps on either of the two resins. When 5b was carried on to the final bicyclic product on the resin, and cleaved to give 10b, the overall yield dropped to 12% over 8 steps. When the peptide alcohols were first cleaved from the resins and then oxidized and cyclized to 10 in solution the overall yield increased to ca. 45% over 8 steps. The major cause for this 4-fold increase in overall yield was the ability to take the oxidation of alcohols 8 to near completion in solution, whereas large amounts of unreacted alcohol peptides were recovered when the oxidation was carried out on the solid phase. The solution phase cyclization of 9 to 10 also demonstrated the effects of sterics on the rate of this second cyclization reaction. Cyclization of 10a was completed within 2 days, whereas 10b took 3 days and 10c took 4 days, all in isopropanol at 50° C. (It is likely that this reaction could be run in much shorter time periods using microwave assisted chemistry.) This suggested that the addition of side chains to the R₁ and R₂ positions helped to restrict the available conformations of the monocyclic intermediate 9 such that the final ring was formed faster. The increased bulk when R₁=Bn (10a) also resulted in a steroselective ring closure to give a 20:1 ratio of diasteromers vs. a 1:1 ratio when R₁═H (10b,c).

### Example 2

### General Method for Removal of Fmoc From Rink Amide MBHA Resin B and Intermediates 2b,c (Scheme 1)

For the series of solid phase reactions on the Rink Amide MBHA Resin described herein, the loading of the particular lot of resin was used to calculate the weight of resin needed to begin with ca. 0.40 mmol of linker sites. All subsequent reactions were run with the full amount of resin obtained in the previous step. Fmoc Rink Amide MBHA resin (513 mg, 0.40 mmol, loading 0.78 mmol/g) was treated with 10 mL piperidine/DMF (¼) for 3 min at room temperature in 20 mL reaction tubes on a Mettler Toledo Bohdan MiniBlock™ parallel reactor station. The reaction mixture was then filtered through the fritted disk at the bottom of the reaction tube and the deprotection treatment was repeated 3 times. The resin was then washed with 3×10 mL of DMF, 3×10 mL of MeOH, and 3×10 mL of DCM. The resin was then dried for two hours in vacuo before carrying out the subsequent coupling reactions. This general procedure was also used for removing the Fmoc group from 2b,c that had been prepared beginning with the same amount of Rink Amide MBHA resin.

### Example 3

### General Method for Coupling of First and Second Amino Acids to Deprotected Rink Amide MBHA Resins 1b and 3b,c (Scheme 1)

The Fmoc-L-amino acid (2 mmol), PyBOP (1.4 g, 5 eq., 2 mmol) and HOBt (0.27 g, 2 mmol) were dissolved in DMF (3 mL). DIEA (0.515 g, 4 mmol) was added to the amino acid solution. The mixture was stirred and the solution was immediately added to the deprotected Rink Amide MBHA resin (1b or 3b,c) prepared as described in Example 2 and in the indicated quantity, in a 20 mL reaction vessel on the MiniBlock reactor. The mixture was stirred overnight. The resin was then washed with 3×10 mL of DMF, 3×10 mL of MeOH, and 3×10 mL of DCM. The resin was then dried for two hours in vacuo before repeating this coupling step again with either the Fmoc-L-amino acid or Fmoc-L-homoserine-O-trityl ether to drive the coupling reaction to completion.

### Example 4

### General Method for Removal of the Trityl Group from Resins 4b,c (Scheme 1)

The dry resin-bound dipeptide 4b,c, prepared as described in Example 3, was pre-swelled with dry DCM (5 mL) for about 30 minutes. Excess DCM was removed by filtering through the reactor fritted disc. DCM/TFA/TIS 94/1/5 (10 mL) was added and the mixture was stirred for 2 min. The reaction solution was then removed by filtration. This reaction with the TFA deprotection mixture was repeated three times, then the resin was washed with DCM (10 mL/each) five times and dried overnight in vacuo at 60° C. to give 5b,c.

### Example 5

### Dess-Martin Oxidation of Resin-Bound Alcohol 5b (Scheme 1)

To a suspension of resin-bound dipeptide alcohol 5b (prepared as described in Example 4) in 9 mL CH₃CN/DMF (2/1) was added Dess-Martin periodinane 15% in DCM (2.26 mL, 0.8 mmol, 2 equiv) and the mixture was stirred overnight at 60° C. The resin was then washed with 3×10 mL of DCM, 3×10 mL of MeOH, 3×110 mL of DCM, and 3×110 mL of THF. The resin was then dried in vacuo at room temperature to give 6b. The resin was subjected to these oxidation conditions twice to drive the reaction closer to completion.

### Example 6

### Bicyclization of Resin-Bound Monocyclic Intermediate 6b (Scheme 1)

The dry resin 6b (prepared as described in Example 5) was pre-swelled with dry DCM (5 mL) for about 30 minutes. Excess DCM was removed by filtering through the reactor fritted disc and then isopropanol (10 mL) was added and the mixture was stirred at 80° C. for four days. The resin was washed with 3×10 mL of DMF, 3×10 mL of MeOH, and 3×10 mL of DCM, and then the resin was dried in vacuo at room temperature for 2 h.

### Example 7

### General Method for Cleavage of 8b,c or 10b From Rink Amide MBHA Resin (Scheme 1)

The products were cleaved from resins 5b,c or 7b (prepared as described in Example 6) by treatment with 10 mL of TFA/TIS/H₂O (94/5/1) for 4 h at room temperature while stirring on the MiniBlock reactor. The product was collected in the cleavage solution by draining through the fritted disk at the bottom of the reaction tube. Cleavage was repeated a second time and then the resin was washed with CH₃CN (3×5 mL). The combined filtrates (including wash solutions) were collected and concentrated in vacuo. The resulting residue was diluted with an ether/hexane mixture (1/1) and kept in the freezer until complete precipitation of the product occurred. The crude product was recovered by filtration and then purified by flash chromatography using the solvent systems indicated below. The flash chromatography solvent systems were generally chosen so that the desired product had a TLC R_{f} of ca. 0.25. The yields indicated below are overall yields calculated from the 0.40 mmols of resin linker sites used in the first step.

TLC and flash chromatography for alcohol 8b: DCM/MeOH 30/1, R_{f}=2.5, (82% yield); ¹H NMR (300 MHz, CDCl₃) δ 1.42 (d, 3H, J=6.9 Hz), 1.82-1.96 (m, 1H), 2.11-2.26 (m, 1H), 3.65-3.76 (m, 2H), 4.18-4.22 (t, 1H, J=6.3), 4.37-4.42 (1, 2H), 4.46-4.51 (t, 2H, J=7.2 Hz), 4.99-5.08 (m, 1H), 5.42-5.55 (b, 1H, exchangeable), 5.68-5.71 (d, 2H, J=7.5 Hz, exchangeable), 7.27-7.32 (m, 3H, 1H), 7.39 (t, 2H, J=7.5 Hz), 7.57 (d, 2H, J=5.1 Hz), 7.75 (d, 2H, J=7.8 Hz), 7.96 (b, 1H, exchangeable). ESI/MS m/z 434 (100) [M+Na], 412 (20) [M+1].

TLC and flash chromatography for alcohol 8c: DCM/MeOH 30/1, R_{f}=2.5, (84% yield); ¹H NMR (400 MHz, Acetone) δ 1.983-1.999 (m, 2H), 3.350-3.405 (m, 2H), 3.689 (d, 1H, J=5.2 Hz), 3.739 (d, 1H, J=5.2), 3.779 (d, 1H, J=6.8 Hz), 3.82 (b, 1H), 4.226-4.324 (m, 3H), 6.401 (b, 1H, exchangeable), 6.483 (b, 1H, exchangeable), 7.049-7.064 (b, 1H, exchangeable), 7.118 (b, 1H, exchangeable), 7.317 (t, 2H, J=7.6 Hz), 7.400 (t, 2H, J=7.6 Hz), 7.710 (t, 2H, J=6.8 Hz), 7.848 (d, 2H, J=8.0 Hz). ESI/MS m/z 817 (100) [Dirner+Na], 420 (10) [M+Na], 398 (3) [M+1].

TLC and flash chromatography for bicyclic structure 10b: DCM/MeOH (35/1), R_{f}=2.5, (68% yield from solution phase synthesis and 12% overall yield from MBHA resin via cleavage of 7b); ¹H NMR (400 MHz, CDCl₃) δ 1.410 (d, 3H, J=5.2 Hz), 2.160-2.200 (m, 1H), 2.86-2.335 (m, 1H), 4.104 (t, 1H, J=7.2 Hz), 4.279 (d, 2H, J=6.0 Hz), 4.286-4.430 (m, 2H), 4.938-5.015 (m, 1H), 6.126 (s, b, 1H, exchangeable), 6.802 (s, 1H, exchangeable), 7.247 (d, 2H, J=5.2 Hz), 7.332 (t, 2H, J=7.2 Hz), 7.539 (t, 2H, J=6.6 Hz), 7.688 (d, 2H, J=7.6 Hz). ESI/MS m/z 391 (20) [M], 392 (5) [M+1], 197 (40) [M-Fmoc-O], 171 (100) [M-Fmoc-OCO]. HRMS (ESI) m/z 392.16032 [(M+Na)⁺, calcd for 392.16103].

### Example 8

### Synthesis of N-Benzyl FMP Resin 1a From Formyl Monomethoxy (FMP) Resin (Scheme 1)

For the series of solid phase reactions on the Formyl MonoMethoxy (FMP) resin described herein, the loading of the particular lot of resin was used to calculate the weight of resin needed to begin with ca. 0.40 mmol of linker sites. All subsequent reactions were run with the full amount of resin obtained in the previous step. A mixture of 2-(4-formyl-3-methoxyphenoxy)methyl polystyrene (FMP resin, 0.444 g, 0.40 mmol, 0.9 mmol/g), 3 mL HC(OEt)₃ (TEOF) and benzyl amine (0.428 g, 4 mmol) in 5 mL 1,2-dichloroethane (DCE) was stirred on the Bohdan reactor station in a 20 mL glass reactor tube with orbital shaking and a slow stream of N₂ gas passing through the mixture for 2 h. The reaction solution was then removed by vacuum filtration through the reaction tube fritted disc, and the resin was charged with 1% AcOH/DMF (10 mL) and then treated with NaBH(OAc)₃ (0.423 g, 2 mmol) while stirring as before, thereby generating a white turbid suspension. Benzyl amine (0.428 g, 4 mmol) was added again and the suspension was stirred well overnight. The reaction solution was then removed from the resin by applying N₂ pressure to the top of the reaction tube with the exit port below the fritted disc open. The resin was then washed with 5×10 mL of MeOH, 5×10 mL of DMF, and 5×10 mL of DCM, and finally once with 10 mL MeOH and then once with 10 mL DCM. The resin was then quickly dried by vacuum filtration through the fritted disk at the bottom of the reaction tube. The 1a resin was used immediately for the coupling reaction described in Example 9. If the resin was not used immediately, the yield in the subsequent coupling reaction was much lower.

### Example 9

### Coupling of L-Ala to Produce 2a (Scheme 1)

The 1a resin (prepared as described in Example 8) was soaked in dry DCM (5 mL) immediately after washing as described above. Fmoc-L-Ala-OH (0.623 g, 2 mmol) and HOBt (0.27 g, 2 mmol) were dissolved in the smallest amount of DMF (ca. 1-2 mL) possible. When larger amounts of DMF were used in this coupling reaction, much lower yields were obtained. DIC (0.505 g, 4 mmol) was added and the mixture was stirred well on the MiniBlock reactor station for 20 minutes at 0° C. (the reactor was fitted with a cooling block) then stirred overnight at room temperature. The reaction was then filtered through the reaction tube fritted disc using N₂ pressure. The resin was washed with 3×10 mL of DMF, 3×10 mL of MeOH, and 3×10 mL of DCM, and then dried by filtration under vacuum. To ensure that complete coupling occurs, the resin was subjected twice to these coupling conditions. After the second coupling the resin was dried in a vacuum dessicator overnight.

### Example 10

### Removal of Fmoc from 2a (Scheme 1)

The resin 2a (prepared as described in Example 9) was treated with 10 mL of piperidine/DMF (1/4) for three minutes while stirring on the MiniBlock reactor; then, the reaction solvent was removed by filtration under N₂ pressure. This deprotection reaction was repeated 3 or 4 times to ensure complete removal of the Fmoc protecting group. The resulting resin 3a was then washed with 3×5 mL of DMF, 3×5 mL of MeOH, and 3×5 mL of DCM, and finally dried in a vacuum dessicator overnight before coupling to Fmoc-L-Hse-O-Tr.

### Example 11

### Coupling of Fmoc-LHse-O-Tr to 3a (Scheme 1)

Fmoc-L-homoserine-O-trityl ether (5 eq. relative to resin loading), PyBOP (5 eq.) and HOBt (5 eq.) were dissolved in 5 mL DMF. DIEA (10 eq. relative to resin loading) in 3 mL DCM was added to the amino acid solution. The mixture was stirred and then the solution was immediately added to the resin 3a (prepared as described in Example 10) pre-swelled in 2 mL of DCM for 45 min with stirring. The mixture was then stirred overnight. After removing the reaction solution by filtration under N₂ pressure, the resin was washed with 3×10 mL of DMF, 3×10 mL of MeOH, and 3×10 mL of DCM. The resulting resin 4a was then dried in a vaccum dessicator overnight before repeating this coupling step again to ensure a high coupling yield.

### Example 12

### Removal of the Trityl Group from 4a (Scheme 1)

The dry resin 4a (prepared as described in Example 11) was pre-swelled with 3 mL of dry DCM for about 45 min with stirring. Excess DCM was removed by gravity filtration though the fritted disc at the bottom of the reaction tube and then 10 mL of DCM/TFA/TIS (94/1/5) was added and the mixture was stirred for 2 min. The reaction solution was then removed by applying N₂ pressure to the top of the reaction tube with the bottom valve opened. This deprotection reaction was repeated on the resin three times to insure complete deprotection. The resin was then washed with DCM (5×10 mL) and the resin was dried by filtration under N₂ pressure to give 5a.

### Example 13

### Cleavage of 8a From the FMP Resin 5a (Scheme 1)

The dipeptide 8a was cleaved from the resin 5a (prepared as described in Example 12) by treatment with 10 mL of TFA/DCM/Et₃SiH (10/30/1) for 1 h. Cleavage was repeated again. The cleavage solutions were collected and the resin was washed with CH₃CN (3×5 mL) to recover any residual trapped product. The combined cleavage solutions and filtrates were collected and concentrated in vacuum. The residue was diluted with ether and kept in the freezer until complete precipitation of the product had occurred. The solid precipitate was recovered by filtration, washed with ether several times, then with DCM/n.hexane mixture (1:1), and finally purified by flash chromatography using the solvent system indicated below.

TLC for alcohol 8a: DCM/MeOH 35/1, R_{f} 0.25, (82% yield); flash chromatography for alcohol 8a: DCM/MeOH (40/1); ¹H NMR (400 MHz, DMSO-d₆): δ 1.233 (d, 3H, J=6.8 Hz), 1.655-1.726 (m, 1H), 1.784-1.884 (m, 1H), 3.429-3.456 (dd, 2H, J=5.2, 5.6 Hz), 4.094 (t, 2H, J=4.8 Hz), 4.157 (d, 2H, J=5.2 Hz), 4.248-4.298 (m, 3H), 4.558 (t, 1H, J=4.8 Hz, exchangeable), 7.205 (t, 3H, J=7.6 Hz), 7.265 (d, 2H, J=6.8 Hz), 7.309 (t, 2H, J=7.2 Hz), 7.380-7.417 (dd, 2H, J=7.2, 7.6 Hz), 7.552 (d, 1H, J=7.6 Hz, exchangeable), 7.681-7.715 (dd, 2H, J=7.2, 6.4 Hz), 7.876 (d, 2H, J=7.6 Hz), 8.006 (d, 1H, J=7.2 Hz, exchangeable), 8.360-8.389 (dd, 1H, J=5.6, 6.0 Hz, exchangeable). ESI/MS m/z 524 (100)[M+Na], 502 (50) [M+1], 279 (20) [M-Fmoc-OCO].

### Example 14

### General Procedure for Dess-Martin Oxidation of 8a,b,c in Solution (Scheme 2)

A solution of the alcohol (8a,b,c, typically ca. 0.2 mmol) was dissolved in CH₃CN (30 mL/g of alcohol). If the alcohol was not completely soluble in this amount of CH₃CN at 50° C., as was the case with 8b,c, then DMF was added dropwise until the alcohol completely dissolved. It was important to use as little DMF as possible, since lower yields typically were obtained when large amounts of DMF were used. Dess Martin (15% solution in DCM, 2 eq) was added and the reaction was run at 50° C. for 1 h. The reaction mixture was then diluted with ether (30 mL) and poured into saturated aqueous NaHCO₃ (20 mL) containing a seven fold excess of Na₂S₂O₃ to quench the unreacted oxidant. The resulting mixture was stirred to dissolve the solid, and the layers were separated. The ether layer was diluted with EtOAc (50 mL) and extracted with saturated NaHCO₃ and then with water. The organic layer concentrated under vacuum and the product was purified by flash chromatography using the solvent system indicated below.

TLC and flash chromatography for monocyclic structure 9a: SiO₂, DCM/MeOH (35/1), (82% yield); ¹H NMR (400 MHz, CDCl₃): δ 1.524 (d, 3H, J=6.8 Hz), 2.243-2.332 (m, 1H), 2.691-2.771 (m, 1H), 4.175-4.219 (m, 2H), 4.276-4.351 (m, 3H), 4.552-4.675 (m, 1H), 4.619-4.675 (m, 1H), 5.259 (b, 1H), 5.765 (d, 1H, J=7.2 Hz, exchangeable), 6.087 (d, 1H, J=7.6 Hz, exchangeable), 7.021 (b, 1H, exchangeable) 7.079-7.141 (m, 2H), 7.177 (d, 2H, J=4.0 Hz), 7.251-7.277 (m, 2H), 7.342-7.391 (m, 2H), 7.415-7.504 (m, 3H), 7.721 (dd, 2H, J=7.2, 4.0 Hz). HRMS (ESI) m/z 522.19946 [(M+Na)⁺, calcd for 522.20049].

TLC and flash chromatography for monocyclic structure 9b: DCM/MeOH 30/1, R_{f}=2.5 (82% yield); ¹H NMR (500 MHz, CDCl₃) δ 1.433 (d, 3H, J=7.0 Hz), 2.231-2.334 (m, 1H), 2.714-2.774 (m, 1H), 4.044-4.103 (m, 2H), 4.253-4.321 (m, 2H), 4.535 (q, 1H, J=7.0 Hz), 5.175-5.239 (b, 1H), 5.586 (b, 1H, exchangeable), 6.334 (s, b, 1H, exchangeable), 6.627-6.643 (m, 1H, exchangeable), 7.006 (s, b, 1H, exchangeable), 7.195-7.219 (m, 2H), 7.301 (q, 2H, J=7.5 Hz), 7.490-7.510 (dd, 2H, J=6.5, 7.0), 7.664 (q, 2H, J=7.0 Hz). ESI/MS m/z 432 (100) [M+Na], 410 (5) [M+1], 392 (50) [M−H₂O]. HRMS (ESI) m/z 432.15341 [(M+Na)⁺, calcd for 432.15354].

TLC and flash chromatography for monocyclic structure 9c: DCM/MeOH 27/1, R_{f}=2.5 (80% yield); ¹H NMR (400 MHz, CDCl₃) δ 2.008 (b, 1H), 2.301 (b, 1H), 3.626-3.639 (m, 1H), 3.860 (d, 1H, J=6.0 Hz), 4.097 (b, 2H), 4.258 (b, 1H), 4.352 (b, 1H), 5.207 (b, 1H), 5.333 (b, 1H, exchangeable), 6.140 (b, 1H, exchangeable), 6.33 (s, b, 1H, exchangeable) 6.991 (d, 1H, exchangeable), 7.197-7.248 (m, 2H), 7.319 (dd, 2H, J=7.6, 7.2 MHz), 7.506 (b, 2H), 7.684 (d, 2H, J=7.6 Hz) ESI/MS m/z 813 (100) [Dimer+Na], 418 (92) [M+Na], 394 (15) [M-1], 378 (55) [M−H₂O].

### Example 15

### General Procedure for Bicyclization of the Monocyclic Products 9a,b,c in Solution (Scheme 2)

The monocyclic alcohol 9a,b,c (typically 0.15 mmol) was dissolved in 15 mL of isopropanol and the mixture was heated to reflux for several days until complete disappearance of the starting monocyclic alcohol was noted by TLC. The products 10a,b,c were then purified by flash chromatography using the solvent systems indicated below.

TLC and flash chromatography for bicyclic structure 10a: DCM/MeOH (40/1), R_{f} 0.25 (75% yield from 9a in Scheme 2). ¹H NMR (400 MHz, CDCl₃): δ 1.533 (d, 3H, J=7.5 Hz), 2.161-2.249 (m, 1H), 2.329-2.419 (m, 1H), 4.169 (t, 1H, J=6.5 Hz), 4.241-4.322 (m, 3H), 4.427 (d, 2H, J=7.5 Hz), 4.651-4.695 (dd, 1H, J=7.5, 7.0 Hz), 4.993 (d, 1H, J=5.5 Hz), 6.264 (d, 1H, J=6.5 Hz, exchangeable), 7.173 (t, 1H, J=6.5 Hz, exchangeable), 7.223-7.252 (m, b, 4H) 7.310-7.341 (m, 2H), 7.420 (t, 2H, J=8.0 Hz), 7.576 (d, 2H, J=6.5 Hz), 7.782 (d, 2H, J=7.5 Hz). HRMS (ESI) m/z 482.20685 [(M+Na)⁺, calcd for 482.20798].

See Example 7 for data for bicyclic structure 10b.

TLC and flash chromatography for bicyclic structure 10c: DCM/MeOH (35/1), R_{f}=0.25, (58% yield from 9c in Scheme 2); ¹H NMR (400 MHz, CDCl₃) δ 2.285-2.473 (m, 2H), 3.506 (q, 1H, J=7.2 Hz), 3.791 (s, 2H), 4.163 (t, 11H, J=6.8 Hz), 4.200 (d, 1H, J=10.4 Hz), 4.349 (d, 1H, J=10.4 Hz), 4.980-5.023 (dd, 1H, J=6.0, 4.4 Hz), 6.016 (d, 1H, J=6.8 Hz, exchangeable), 7.060 (b, 1H, exchangeable), 7.294 (t, 2H, J=7.4 Hz), 7.387 (t, 2H, J=7.4 Hz), 7.516-7.565 (dd, 2H, J=7.6, 4.0 Hz), 7.743 (d, 2H, J=7.6 Hz). ESI/MS m/z 378 (100) [M+Na].

### Example 16

### An Alternative Complete Solution Phase Route to Tetrahydro-pyrrolo[1,2-a]imidazole-2,5-dione Derivatives

A full solution phase synthesis of the dipeptide or tripeptide intermediates is an alternative to the solid phase route outlined in the previous examples. After several optimizations using various coupling reagents, the mixed anhydride coupling method, modified by adding a catalytic amount of dimethylaminopyridine (DMAP), was found to produce the dipeptide and tripeptide intermediates in good yields. The main problem with this route was that the detritylation step (5 to 6, Scheme 3 below) is not a good step, due to the side products produced that need to be removed by chromatography. This problem makes the solution phase parallel synthesis of dipeptides, such as 6, more difficult to implement because chromatographic purification of the deprotected dipeptides may be needed before going on to the next step. Therefore, a new route (Scheme 4 below) was investigated for the synthesis of the dipeptides 6 & 12. Efforts to convert benzyl esters 10 to the alcohols 12 as indicated in Scheme 4 (below) were promising and this approach may provide a solution to the detritylation step problem above. Scheme 5 below details an example of the general synthesis outlined in Scheme 3 wherein R₂ is an amino acid amide.

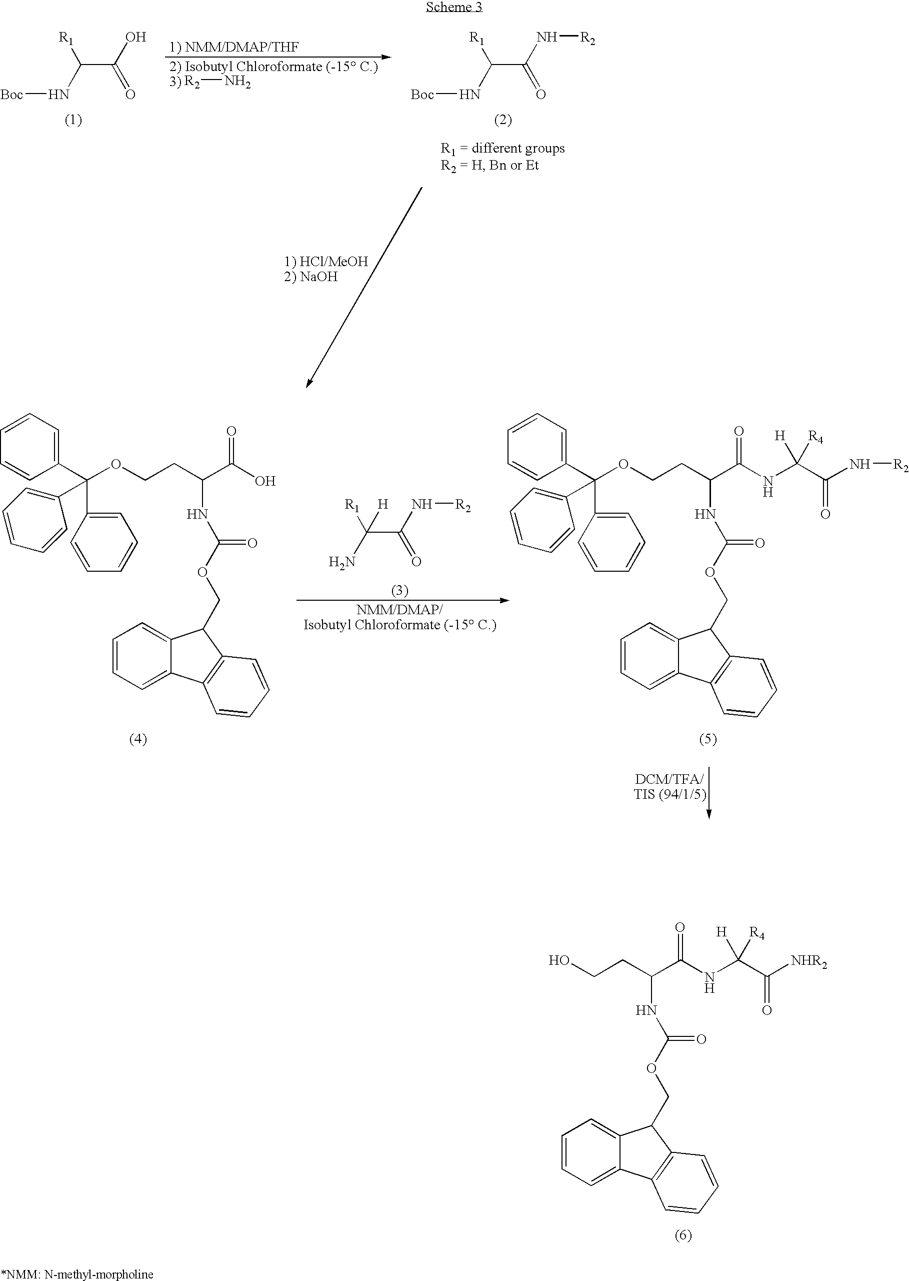

### Example 17

### General Procedure for the Preparation of N-Boc-α-amino Amides from N-Boc-α-amino Acids

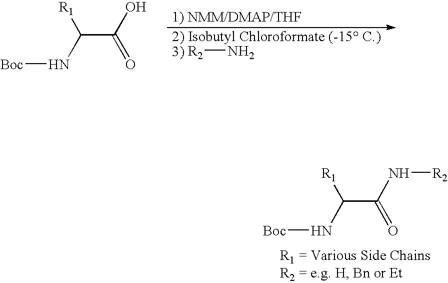

To a cold solution (−15° C.) of N-Boc-α-amino acid (10 mmol) and 4-methylmorpholine (1.10 g, 10 mmol) in dry THF (30 mL) was added dropwise isobutyl chloroformate (1.36 g, 10 mmol) in THF (5 mL) over 15 min. After 5 min of stirring at −15° C., DMAP (0.122 g, 1.0 mmol) in dry THF (5 mL) was added into the reaction mixture. After stirring for another 15 min, an appropriate primary amine (10 mmol) was added in one portion. Then, the reaction mixture was allowed to warm to room temperature and stirred overnight. After evaporation of the solvent in vacuo, the residue was diluted with EtOAc and the organic phase was washed with 10% Na₂CO₃, 0.1 M HCl, brine and dried over anhydrous Na₂SO₄. Removal of the solvent in vacuo gave crude N-Boc-α-amino amides which can be used for the subsequent step without further purification. For amino acid amide HCl salts, 2 equiv. of 4-methylmorpholine (2.02 g, 20 mmol) were used. For further purification, the crude solid was crystallized from CHCl₃-hexanes.

### Example 18

### General Procedure for the Preparation of α-amino Amides from N-Boc-α-amino Amides

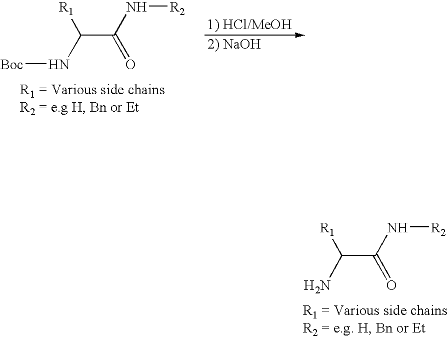

To a stirred solution of N-Boc amino amides (2.2 mmol) in EtOAc (10 mL), HCl in MeOH (ca. 3M, 10 mL) was added. The mixture was stirred at room temperature until a TLC analysis showed the disappearance of the starting material (10-12 h). The mixture was evaporated in vacuo and the solid so obtained was washed with ether several times. After drying, the product was characterized by spectral data. (Primary aminoacid amides showed poor solubility in organic solvents and extraction will affect their yields dramatically.)

For N-alkyl amino acid amides (e.g., N-ethyl or N-benzyl amino acid amides), the mixture was treated with 1 M NaOH and extracted with EtOAc. The organic phase was washed with brine and dried over anhydrous Na₂SO₄. Evaporation of solvent in vacuo gave the α-amino amides, which were recrystallized from appropriate solvents (if solid) or purified by column chromatography with hexane-EtOAc as eluent (if oil).

For example, the preparation of 2-amino-N-benzyl-propionamide is shown below: 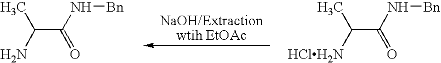
(Crude, 80% yield, ca. 100% pure), ¹H NMR (400 MHz, CDCl₃) δ 1.320 (d, 3H, J=4.8 Hz), 1.810 (b, 1H, exchangeable), 3.473-3.526 (m, 1H), 4.386-4.401 (AB quartet, 2H), 7.20-7.246 (m, 3H), 7.279-7.315 (m, 2H), 7.65 (b, 1H, exchangeable).

LRMS (ESI) m/z 179 (100) [M+H].

### Example 19

### General Procedure for the Preparation of Homoserine Dipeptides

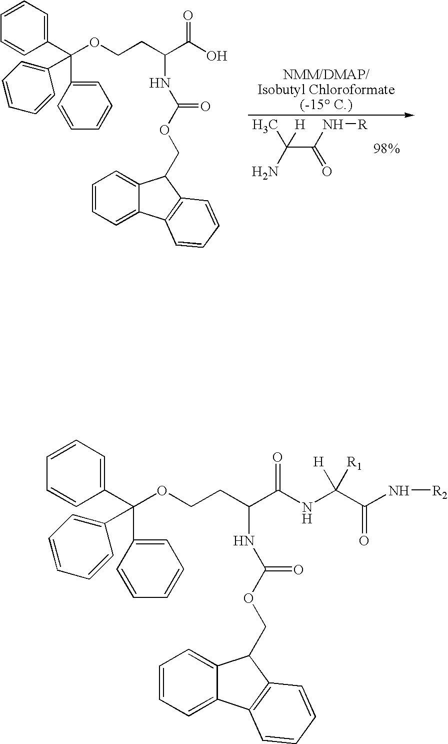

To a cold solution (−15° C.) of Fmoc-L-Hse-OTr (1.5 mmol), 4-methylmorpholine (1.5 mmol) in dry THF (100 mL), isobutyl chloroformate (1.5 mmol) in THF (2 mL) was added dropwise over 15 min. After 5 min of stirring at −15° C., DMAP (0.15 mmol) in dry THF (2 mL) was added to the reaction mixture. After stirring for another 15 min, an appropriate amino acid amide (1.5 mmol) was added in one portion. Then, the reaction mixture was allowed to warm to room temperature and stirred overnight. After evaporation of the solvent in vacuo, the residue was diluted with EtOAc and the organic phase was washed with 10% Na₂CO₃, 0.1 M HCl, brine and dried over anhydrous Na₂SO₄. Removal of the solvent in vacuo gave crude N-Boc dipeptide amides which can be used for the subsequent step without further purification. For amino acid amide HCl salts, 2 equiv. of 4-methylmorpholine (3 mmol) were used. For further purification the crude solid was crystallized from CHCl₃-hexanes. (crude, 98% yield, 80% after crystallization), ¹H NMR (400 MHz, CDCl₃) δ 1.230 (d, 3H, J=6.8 Hz), 1.793-1.829 (m, 1H), 2.02-2.073 (m, 1H), 2.961 (m, 1H), 3.022-3.063 (m, 1H), 4.128-4.4163 (m, 1H), 4.217 (s, b, 2H), 4.256-4.302 (m, 3H), 7.201 (d, 4H, J=6.8 Hz), 7.25-7.284 (m, 12H), 7.342-7.393 (m, 8H), 7.553 (d, 1H, J=8.4 Hz, exchangeable), 7.652 (b, 2H), 7.873 (d, 2H, J=6.4 Hz), 8.156 (d, 1H, J=7.6 Hz, exchangeable), 8.369 (b, 1H, exchangeable).

LRMS (ESI) m/z 766 (4) [M+Na], 524 (4) [M-Trityl+Na], 502 (3) [M+Na] [M-Trityl], 279 (100)[M-Trityl-Fmoc].

The following reaction shows a specific example of an amino acid being coupled to protected homoserine.

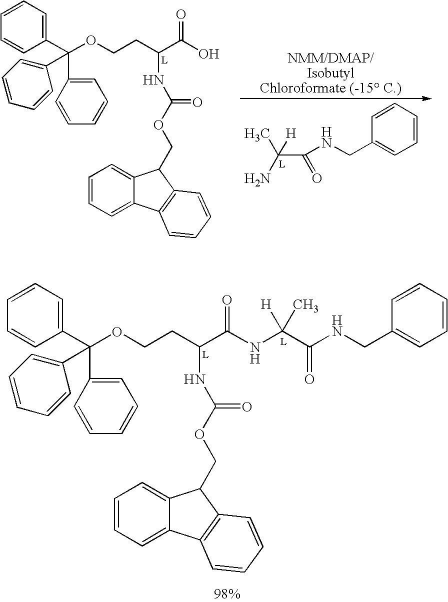

### Example 20

### General Procedure for Cleavage of Trityl Group

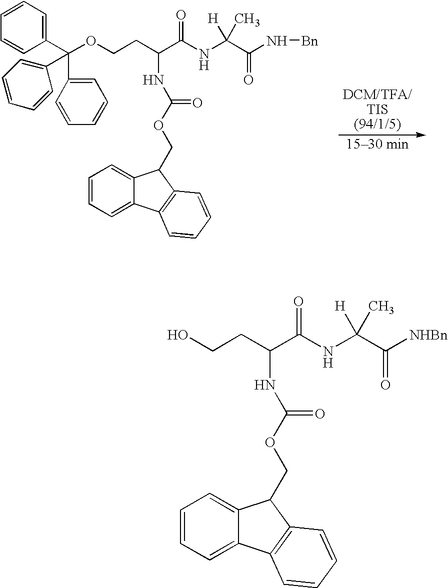

To a solution of the dipeptide-OTr (1.5 mmol) in DCM (10 mL), a mixture of DCM/TFA/TIS (40 mL) was added in a ratio of 94:1:5 and the mixture was stirred for 5-15 min. The mixture was evaporated in vacuo and the solid precipitate was chromatographed using a silica gel column. Compound 6 from Scheme 3, where R₁═CH₃, R₂=benzyl: (SiO₂, DCM/MeOH 35:1), (68% yield). ¹H NMR (400 MHz, DMSO, d₆): δ 1.233 (d, 3H, J=6.8 Hz), 1.656-1.726 (m, 1H), 1.784-1.884 (m, 1H), 3.443 (dd, b, 1H, J=5.6, 5.2 Hz), 4.082-4.106 (m, 2H), 4.157 (d, 2H, J=5.2 Hz), 4.248-4.298 (m, 3H), 4.558 (t, 1H, J=4.8 Hz, exchangeable), 7.195 (t, 3H, J=7.6 Hz), 7.256-7.273 (m, 2H, J=7.2, 6.8 Hz), 7.318 (d, 2H, J=7.2 Hz), 7.380-7.417 (m, 2H), 7.552 (d, 1H, J=7.6 Hz, exchangeable), 7.681-7.715 (m, 2H), 7.876 (d, 2H, J=7.6 Hz), 8.006 (d, 1H, J=7.2 Hz exchangeable), 8.360-8.389 (m, 1H, exchangeable).

LRMS (ESI) m/z 502 (50) [M+H], 524 (100) [M+Na]

### Example 21

### An Alternative Scheme for Synthesis of the Dipeptides

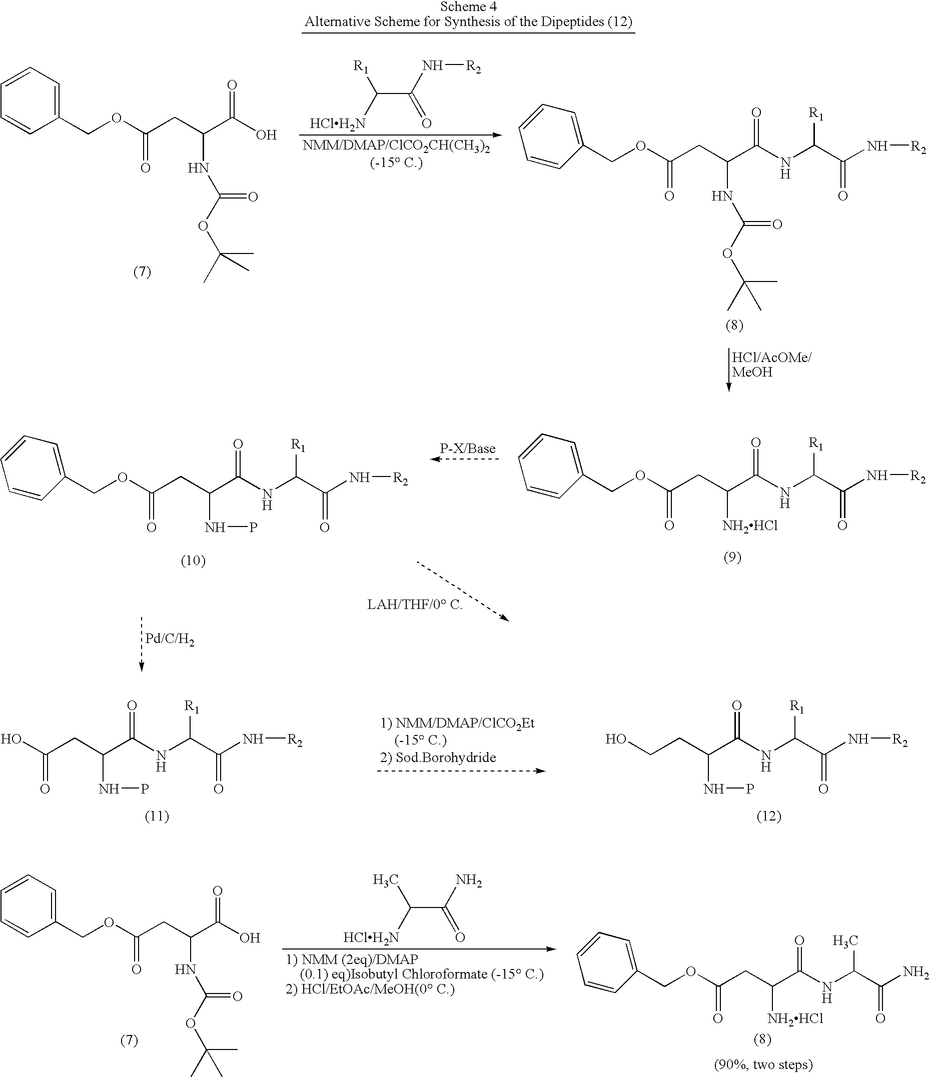

To a cold solution (−15° C.) of N-Boc-β-Benzyl L-aspartate (5.0 mmol), 4-methylmorpholine (10 mmol) in dry THF (100 mL), isobutyl chloroformate (5.0 mmol) in THF (2 mL) was added dropwise over 15 min. After 5 min of stirring at −15° C., DMAP (0.5 mmol) in dry THF (2 mL) was added to the reaction mixture. After stirring for another 15 min, L-alaninamide HCl (5.0 mmol) was added in one portion. Then, the reaction mixture was allowed to warm to room temperature and stirred overnight. After evaporation of the solvent in vacuo, the residue was diluted with EtOAc and the organic phase was washed with 10% Na₂CO₃, 0.1 M HCl, brine and dried over anhydrous Na₂SO₄. Removal of the solvent in vacuo gave crude N-Boc-dipeptide amide which was diluted with EtOAc (20 ml) and then HCl in MeOH (ca. 3 M, 20 mL) was added. The mixture was stirred at 0° C. for half an hour and then at room temperature until a TLC analysis showed the disappearance of the starting material (10-12 h). The mixture was evaporated in vacuo and the solid so obtained was washed with ether several times. After drying, the product was characterized by spectral data. (Crude, 90% yield). ¹H NMR (400 MHz, DMSO, d₆): δ 1.236 (d, 2H, J=5.6 Hz), 2.817-2.887 (m, 1H), 2.994-3.037 (m, 1H), 4.162 (b, 1H), 4.211 (q, 1H, J=5.6 Hz), 5.140 (s, 2H), 7.094 (s, 1H, exchangeable), 7.305 (s, 1H, exchangeable), 7.338-7.388 (m, 5H), 8.365 (m, 3H, exchangeable), 8.698 (d, 1H, exchangeable).

### Example 22

### Solution Phase Synthesis of Dipeptide and Tripeptide Intermediates

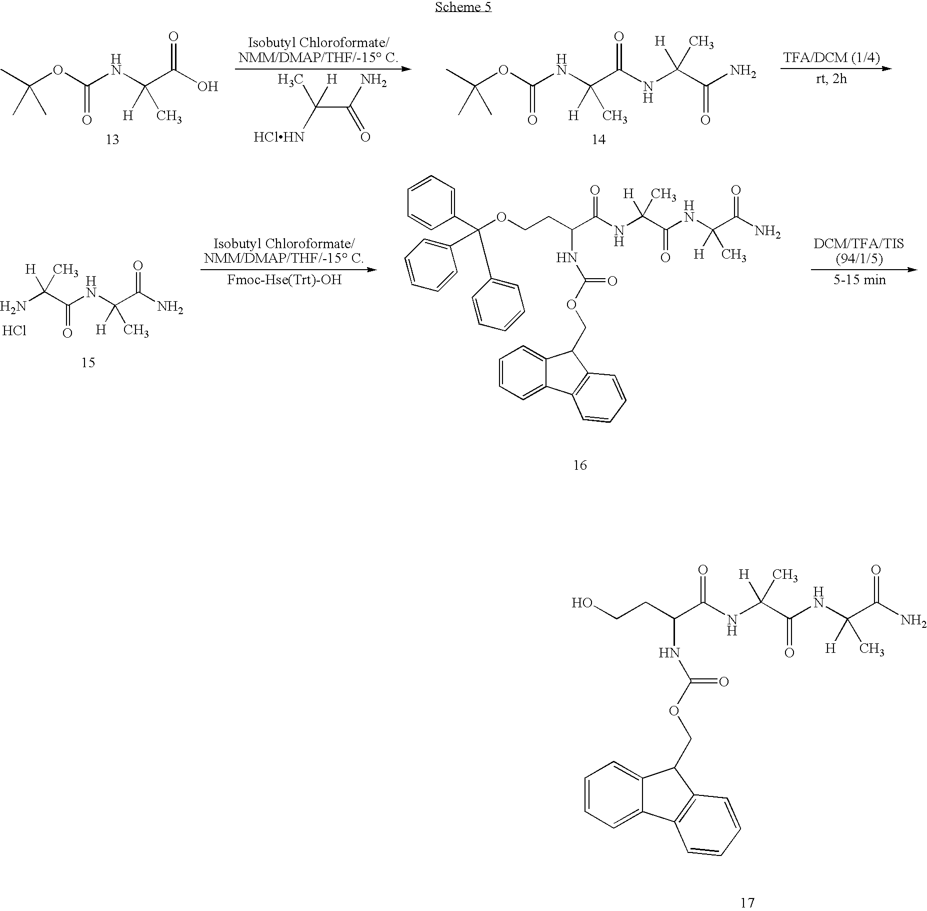 Synthesis of N-Boc-L-Ala-L-Ala-NH₂

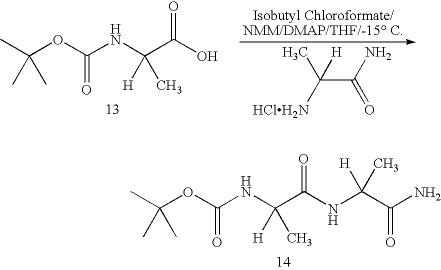

To a cold solution (−15° C.) of N-Boc-L-alanine (10 mmol), 4-methylmorpholine (20 mmol) in dry THF (50 mL), isobutyl chloroformate (10 mmol) in THF (10 mL) was added dropwise over 15 min. After 5 min of stirring at −15° C., dimethylaminopyridine (2.0 mmol) in dry THF (10 mL) was added to the reaction mixture. After stirring for another 15 min, L-alaninamide HCl (10 mmol) was added in one portion. Then, the reaction mixture was allowed to warm to room temperature and stirred overnight. After evaporation of the solvent in vacuo, the residue was diluted with EtOAc and the organic phase was washed with 10% Na₂CO₃, 0.1 M HCl, brine and dried over anhydrous Na₂SO₄. Removal of the solvent in vacuo gave crude N-Boc-L-Ala-L-Ala-NH₂ amide 14 which can be used for the subsequent step without further purification. For further purification the crude solid can be crystallized from CHCl₃-hexanes. Crude, 87% yield. ¹H NMR (300 MHz, DMSO, d₆): δ 1.127 (d, 3H, J=7.2 Hz), 1.163 (d, 3H, J=7.2 Hz), 1.343 (s, 9H), 3.863-3.911 (m, 1H), 4.121-4.169 (m, 1H), 7.00 (s, 2H, exchangeable), 7.264 (s, 1H), 7.707 (d, 1H, J=7.5 Hz).
Removal of Boc Group—Synthesis of the Dipeptide 15

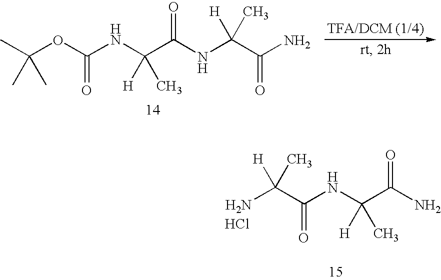

To a stirred solution of N-Boc dipeptide amide 14 (4.6 mmol) in DCM (10 mL), TFA in DCM (1:1) (30 mL) was added. The mixture was stirred for two hours. The mixture was evaporated in vacuo and the solid so obtained was washed with ether several times. After drying the product was characterized by spectral data. (Crude, 94%, ca. 100% pure). ¹H NMR (300 MHz, DMSO, d₆): δ 1.231 (d, 3H, J=7.2 Hz), 1.317 (d, 3H, J=7.2 Hz), 3.823-3.852 (m, 1H), 4.240-4.276 (m, 1H), 7.130 (s, 1H, exchangeable), 7.494 (s, 1H, exchangeable), 8.092 (s, 3H, exchangeable), 8.547 (d, 1H, J=7.6 Hz, exchangeable).
Synthesis of the Tripeptide 16

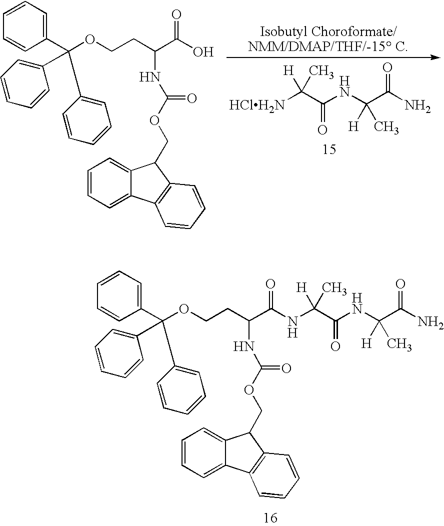

To a cold solution (−15° C.) of the N-Fmoc-O-trityl-L-homoserine (2.3 mmol), 4-methylmorpholine (4.6 mmol) in dry THF (30 mL), isobutyl chloroformate (2.3 mmol) in THF (5 mL) was added over 15 min. After 5 min of stirring at −15° C., dimethylaminopyridine (0.23 mmol) in dry THF (5 mL) was added to the reaction mixture. After stirring for another 15 min, L-Ala-L-Ala-dipeptide amide 15 (2.3 mmol) was added in one portion. Then, the reaction mixture was allowed to warm to room temperature and stirred overnight. After evaporation of the solvent in vacuo, the residue was diluted with EtOAc and the organic phase was washed with 10% Na₂CO₃, 0.1 M HCl, brine and dried over anhydrous Na₂SO₄. Removal of the solvent in vacuo gave crude tripeptide amide 16 which can be used for the subsequent step without further purification. (Crude, 87% yield, over 90% purity). ¹H NMR (300 MHz, DMSO, d₆): δ 1.190 (dd, 6H, J=6.8, 6.4 Hz), 1.720-1.960 (m, b, 1H), 1.85-2.06 (m, b, 1H), 2.92-2.98 (m, b, 1H), 3.00-3.08 (m, b, 1H), 4.138-4.173 (m, 2H), 4.205-4.269 (m, 4H), 6.993 (s, 1H), 7.195 (t, 2H, J=7.2 Hz), 7.235-7.291 (m, 10H), 7.343-7.417 (m, 8H), 7.504 (d, 1H, J=8.0 Hz, exchangeable), 7.647-7.674 (m, 2H), 7.828 (d, 1H, J=7.2 Hz, exchangeable), 7.879 (d, 2H, J=7.6 Hz), 8.099 (d, 1H, J=7.6 Hz, exchangeable).
Synthesis of the Tripeptide 17

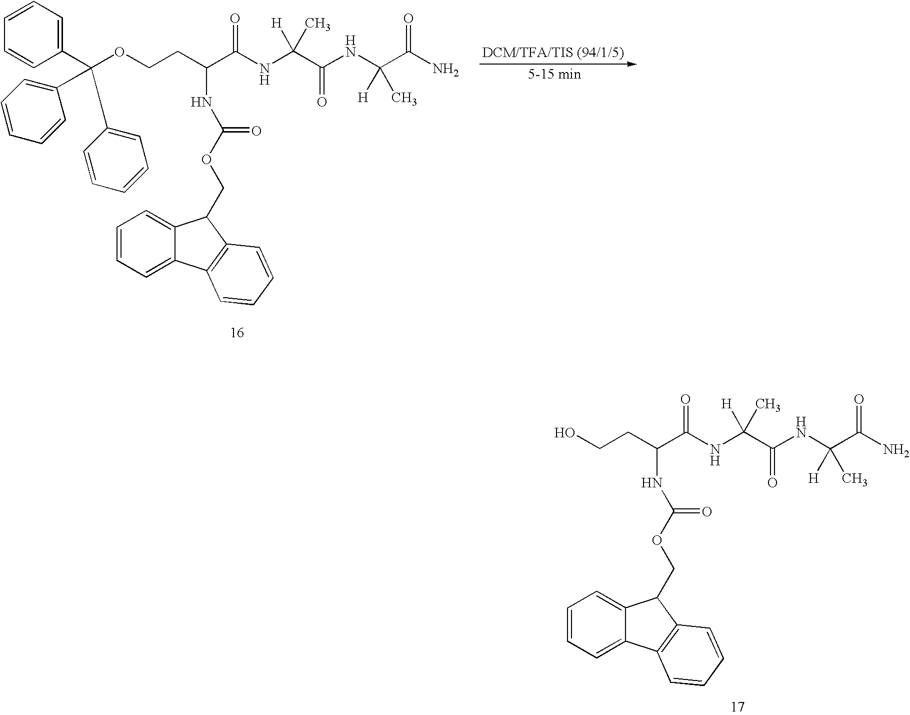

To a solution of the tripeptide-OTr 16 (1.5 mmol) in DCM (10 mL) was added a mixture of DCM/TFA/TIS (40 mL, ratio of 94:1:5) and the mixture was stirred for 5-15 min. The mixture was evaporated in vacuo and the solid precipitate was chromatographed using a silica gel column. (SiO₂, DCM/MeOH 20/1˜5/1˜10/1), (52% yield). ¹H NMR (300 MHz, DMSO, d₆): δ 1.206 (dd, 6H, J=8.5, 7.0 Hz), 1.668-1.735 (m, 1H), 1.810-1.880 (m, 1H), 3.279-3.346 (m, 2H), 3.415-3.510 (m, 2H), 4.094-4.121 (m, 1H), 4.141-4.184 (m, 1H), 4.205-4.271 (m, 2H), 4.544 (t, b, 1H, exchangeable), 6.991 (s, 1H, exchangeable), 7.231 (s, 1H, exchangeable), 7.338 (t, 2H, J=7.5 Hz), 7.426 (t, 2H, J=7.5 Hz), 7.538 (d, 2H, J=8.0 Hz, exchangeable), 7.728 (t, 2H, J=7.5 Hz), 7.841 (d, 2H, J=7.5 Hz, exchangeable), 7.900 (d, 2H, J=7.5 Hz, exchangeable), 8.012 (d, 2H, J=7.0 Hz, exchangeable).

Although the invention has been described in detail, for the purpose of illustration, it is understood that such detail is for that purpose and variations can be made therein by those skilled in the art without departing from the spirit and scope of the invention which is defined by the following claims.

1. A combinatorial library comprising two or more tetrahydro-pyrrolo[1,2-a]imidazole-2,5-dione derivative compounds of the following formula: 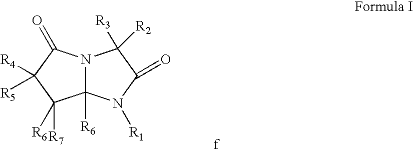
wherein:
R—R₈ are individually selected from the group consisting of: H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein any of R₁-R₈ can be further substituted with one or more halogen atoms or functional groups selected from the group consisting of: amides, ureas, ketones, sulfonamides, phosphoramides, alcohols, thiols, esters, amines, amidines, guanidines, carboxylic acids, sulfonic acids, phosphonic acids, and boronic acids;
R₄-R₇ can optionally be side chains with N, S, O, P, or B directly attached to ring of Formula I, substituted with any combination of H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein the directly attached N, S, or O can also be further attached to a carbonyl, sulfonyl, sulfinyl, or phosphoryl group to form a functional group selected from the group consisting of urea, amide, urethane, sulfonamides sulfmamide, sulfonylurea, sulfinylurea, and phosphoramide, wherein the functional group can be further substituted with H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl, wherein all of the side chains with N, S, O, P, or B can optionally contain one or more halogen atoms and a functional group selected from the group consisting of amide, urea, ketone, sulfonamide, phosphoramide, alcohol, thiol, ester, amine, amidine, guanidine, carboxylic acid, sulfonic acid, phosphonic acid, and boronic acid;
any combination of two substituents selected from R₁-R₈ can be covalently attached to each other to form another ring;
wherein the library comprises enantiomers, diastereomers, or a mixture of the enantiomers and the diastereomers of the compound;
with the proviso that the following conditions cannot all be true at the same time:
R₄═H, R₅═H, R₆=H, and R₇═H.

2. The combinatorial library according to claim 1, wherein one or more of P4-R₇ are side chains with an attached wherein the attached N is a part of a functional group selected from the group consisting of amide, urea, urethane, amidine, guanidine, sulfonamide, sulfonylurea, phosphoramide, unsubstituted amine, mono-alkyl amine, aryl amine, heteroaryl amine, dialkyl amine, and diaryl amine, wherein one or both aryl groups can be a heteroaryl group, wherein the functional group can be further substituted with H, C₁-C₁₂ alkyl heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl.

3. The combinatorial library according to claim 1, wherein the library includes at least one compound having the following formula:

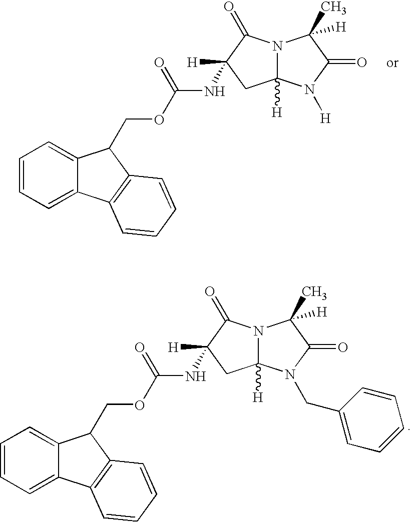

4. A combinatorial library comprising two or more hexahydro-pyrrolo[1,2-a]pyrimidine-2,6-dione derivative compounds of the following formula: 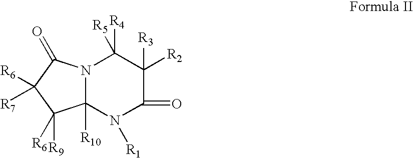
wherein:
R₁-R₁₀ are individually selected from the group consisting of: H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, alkylheteroaryl, and substituted alkylheteroaryl, wherein any of R₁-R₁₀ can be further substituted with one or more halogen atoms or functional groups selected from the group consisting of: amides, ureas, ketones, sulfonamides, phosphoramides, alcohols, thiols, esters, amines, amidines, guanidines, carboxylic acids, sulfonic acids, phosphonic acids, and boronic acids;
R₆-R₉ can optionally be side chains with N, S, O, P, or B directly attached to ring of Formula I, substituted with any combination of H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein the directly attached N, S, or O can also be further attached to a carbonyl, sulfonyl, sulfinyl, or phosphoryl group to form a functional group selected from the group consisting of urea, amide, urethane, sulfonamides sulfmamide, sulfonylurea, sulfinylurea, and phosphoramide, wherein the functional group can be further substituted with H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl, wherein all of the side chains with N, S, O, P, or B can optionally contain one or more halogen atoms and a functional group selected from the group consisting of amide, urea, ketone, sulfonamide, phosphoramide, alcohol, thiol, ester, amine, amidine, guanidine, carboxylic acid, sulfonic acid, phosphonic acid, and boronic acid;
any combination of two substituents selected from R₁-R₁₀ can be covalently attached to each other to form another ring;
wherein the library comprises enantiomers, diastereomers, or a mixture of the enantiomers and the diastereomers of the compound.

5. The combinatorial library according to claim 4, wherein one or more of R₆-R₉ are side chains with an attached wherein the attached N is a part of a functional group selected from the group consisting of amide, urea, urethane, arnidine, guanidine, sulfonamide, sulfonylurea, phosphoramide, unsubstituted amine, mono-alkyl amine, aryl amine, heteroaryl amine, dialkyl amine, and diaryl amine, wherein one or both aryl groups can be a heteroaryl group, wherein the functional group can be further substituted with H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl aryl and substituted aryl, heteroaryl and substituted heteroaryl, alkylaryl and substituted alkylaryl, or alkylheteroaryl and substituted alkylheteroaryl.

6. The combinatorial library according to claim 4, wherein the library includes at least one compound having Formula (II), wherein
R₁═R₂═R₃═R₄═R₅═R₇═R₈═R₉═R₁₀=H and R₆=FmocNH.

7. A tetrahydro-pyrrolo[1,2-a]imidazole-2,5-dione derivative compound of the following formula: 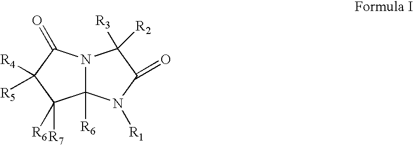
wherein:
R₁-R₈ are individually selected from the group consisting of: H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein any of R₁-R₈ can be further substituted with one or more halogen atoms or functional groups selected from the group consisting of: amides, ureas, ketones, sulfonamides, phosphoramides, alcohols, thiols, esters, amines, amidines, guanidines, carboxylic acids, sulfonic acids, phosphonic acids, and boronic acids;
R₄-R₇ can optionally be side chains with N, S, O, P, or B directly attached to ring of Formula I, substituted with any combination of H, C₁-C₁₂ alkyl heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein the directly attached N, S, or O can also be further attached to a carbonyl, sulfonyl, sulfinyl, or phosphoryl group to form a functional group selected from the group consisting of urea, amide, urethane, sulfonamides sulfmamide, sulfonylurea, sulfinylurea, and phosphoramide, wherein the functional group can be further substituted with H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl, wherein all of the side chains with N, S, O, P, or B can optionally contain one or more halogen atoms and a functional group selected from the group consisting of amide, urea, ketone, sulfonamide, phosphoramide, alcohol, thiol, ester, amine, amidine, guanidine, carboxylic acid, sulfonic acid, phosphonic acid, and boronic acid;
any combination of two substituents selected from R₁-R₈ can be covalently attached to each other to form another ring;
wherein the compound comprises an enantiomer, a diastereomer, a mixture of the enantiomer and the diastereomer, or a pharmaceutically acceptable salt thereof;
with the proviso that any of the following conditions cannot all be true at the same time:
R₄═H, R₅═H, R₆=H, and R₇═H;
R₁═R₃═R₅═R₆═R₇═R₈=H, R₂=isobutyl, and R₄=BocNH;
R₁═R₂═R₅═R₆═R₇═R₈=H, R₃=isobutyl, and R₄=BocNH;
R₁═R₃═R₄═R₆═R₇═R₈=H, R₂=isobutyl, and R₅=BocNH; or
R₁═R₂═R₄═R₆═R₇═F8=H, R₃=isobutyl, and R₅=BocNH.

8. The compound according to claim 7, wherein one or more of R₄-R₇ are side chains with an attached wherein the attached N is a part of a functional group selected from the group consisting of amide, urea, urethane, amidine, guanidine, sulfonamide, sulfonylurea, phosphoramide, unsubstituted amine, mono-alkyl amine, aryl amine, heteroaryl amine, dialkyl amine, and diaryl amine, wherein one or both aryl groups can be a heteroaryl group, wherein the functional group can be further substituted with H, C₁-C₁₂ alkyl heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl.

9. The compound according to claim 7, wherein the compound has the following formula:

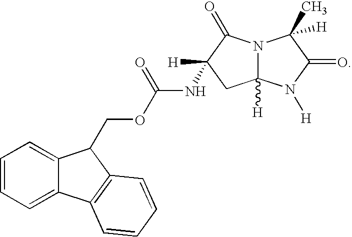

10. The compound according to claim 7, wherein the compound has the following formula:

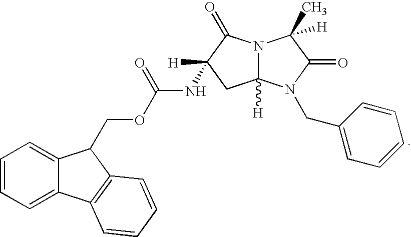

11. A hexahydro-pyrrolo[1,2-a]pyrimidine-2,6-dione derivative compound of the following formula: 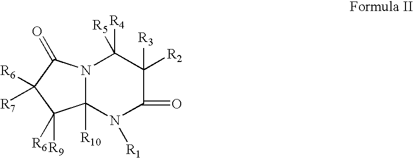
wherein:
R₁-R₁₀ are individually selected from the group consisting of: H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, alkylheteroaryl, and substituted alkylheteroaryl, wherein any of R₁-R₁₀ can be further substituted with one or more halogen atoms or functional groups selected from the group consisting of: amides, ureas, ketones, sulfonamides, phosphoramides, alcohols, thiols, esters, amines, amidines, guanidines, carboxylic acids, sulfonic acids, phosphonic acids, and boronic acids;
R₆-R₉ can optionally be side chains with N, S, O, P, or B directly attached to ring of Formula I, substituted with any combination of H, C₁-C₁₂ alkyl heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein the directly attached N, S, or O can also be further attached to a carbonyl, sulfonyl, sulfinyl, or phosphoryl group to form a functional group selected from the group consisting of urea, amide, urethane, sulfonamides sulfmamide, sulfonylurea, sulfinylurea, and phosphoramide, wherein the functional group can be further substituted with H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl, wherein all of the side chains with N, S, O, P, or B can optionally contain one or more halogen atoms and a functional group selected from the group consisting of amide, urea, ketone, sulfonamide, phosphoramide, alcohol, thiol, ester, amine, amidine, guanidine, carboxylic acid, sulfonic acid, phosphonic acid, and boronic acid;
any combination of two substituents selected from R₁-R₁₀ can be covalently attached to each other to form another ring;
wherein the compound comprises an enantiomer, a diastereomer, or a mixture of the enantiomer and the diastereomer, or a pharmaceutically acceptable salt thereof.

12. The compound according to claim 11, wherein one or more of R₆-R₉ are side chains with an attached wherein the attached N is a part of a functional group selected from the group consisting of amide, urea, urethane, amidine, guanidine, sulfonamide, sulfonylurea, phosphoramide, unsubstituted amine, mono-alkyl amine, aryl amine, heteroaryl amine, dialkyl amine, and diaryl amine, wherein one or both aryl groups can be a heteroaryl group, wherein the functional group can be further substituted with H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl.

13. The compound according to claim 11, wherein
R₁═R₂═R₃═R₄═R₅═R₇═R₈═R₉═R₁₀=H and R₆=FmocNH.

14. A method of preparing a tetrahydro-pyrrolo[1,2-a]imidazole-2,5-dione derivative compound, said method comprising:
providing a functionalized resin solid support;
reacting the functionalized resin solid support with amino acids under conditions effective to produce a resin bound dipeptide or tripeptide alcohol intermediate compound;
oxidizing the resin bound dipeptide or tripeptide alcohol intermediate compound under conditions effective to convert the resin bound dipeptide or tripeptide alcohol intermediate compound to a resin bound dipeptide or tripeptide aldehyde intermediate compound; and
cyclizing the resin bound dipeptide or tripeptide aldehyde intermediate compound under conditions effective to produce the tetrahydro-pyrrolo[1,2-a]iimidazole-2,5-dione derivative compound.

15. The method according to claim 14, wherein the resin bound dipeptide or tripeptide alcohol intermediate is cleaved from the solid support and said oxidizing and said cyclizing are performed in solution.

16. The method according to claim 15, wherein the resin bound dipeptide or tripeptide alcohol intermediate is cleaved from the solid support by using trifluoroacetic acid/dichloromethane/triethyl silane or trifluoroacetic acid/triisopropylsilane/H₂O.

17. The method according to claim 14, wherein the functionalized resin solid support is an amine resin.

18. The method according to claim 17, wherein the amine resin is prepared by treating a formyl monomethoxy (FMP) resin or a rink amide (MBHA) resin.

19. The method according to claim 17, wherein the amine resin is a substituted amine resin.

20. The method according to claim 17, wherein the amine resin is an unsubstituted amine resin.

21. The method according to claim 14, wherein said reacting involves a first coupling step wherein a first amino acid is coupled to the functionalized resin solid support under conditions effective to produce a resin bound amino acid intermediate compound and a second coupling step wherein a second amino acid is coupled to said resin bound amino acid intermediate compound under conditions effective to produce a resin bound dipeptide alcohol intermediate compound.

22. The method according to claim 21, wherein the first amino acid is a Fmoc-L-amino acid or a Fmoc-D-amino acid.

23. The method according to claim 21, wherein the first coupling step is carried out by using a coupling reagent selected from the group consisting of: O-(7-azabenzotriazol-1-yl)-1,1,3,3-tetramethyluronium hexafluorophosphate, 0-(benzotriazol-1-yl)-1,1,3,3-tetramethyluronium hexafluorophosphate, 0-(benzotriazol-1-yl)-1,1,3,3-tetramethyluronium tetrafluoroborate, 1,3-diisopropylcarbodiimide, 1-(3-dimethylaminopropyl)-3-ethylcarbodiimide hydrochloride, 1-benzotriazolyloxytris(pyrrolidino)phosphonium hexafluorophosphate, and bromotris(pyrrolidino)phosphonium hexafluorophosphate.

24. The method according to claim 23, wherein the coupling reagent is 1,3-diisopropylcarbodiimide or 1-benzotriazolyloxytris(pyrrolidino)phosphonium hexafluorophosphate.

25. The method according to claim 21, wherein the second coupling step is carried out by using a coupling reagent selected from the group consisting of: O-(7-azabenzotriazol-1-yl)-1,1,3,3-tetramethyluronium hexafluorophosphate, O-(benzotriazol-1-yl)-1,1,3,3-tetramethyluronium hexafluorophosphate, O-(benzotriazol-1-yl)-1,1,3,3-tetramethyluronium tetrafluoroborate, 1,3-diisopropylcarbodiimide, 1-(3-dimethylaminopropyl)-3-ethylcarbodiimide hydrochloride, 1-benzotriazolyloxytris(pyrrolidino)phosphonium hexafluorophosphate, and bromotris(pyrrolidino)phosphonium hexafluorophosphate.

26. The method according to claim 25, wherein the coupling reagent is 1-benzotriazolyloxytris(pyrrolidino)phosphonium hexafluorophosphate.

27. The method according to claim 14, wherein said oxidizing is carried out by using an oxidation reagent selected from the group consisting of:
Swern, pyridine-SO₃, tetra-n-propylammonium perruthenate(VII), pyridinium chlorochromate, pyridinium dichromate, and Dess-Martin periodinane.

28. The method according to claim 27, wherein the oxidation reagent is Dess-Martin periodinane.

29. The method according to claim 14, wherein said cyclizing is carried out under alcohol refluxing conditions.

30. The method according to claim 14, wherein the tetrahydro-pyrrolo[1,2-a]imidazole-2,5-dione derivative compound has the following formula: 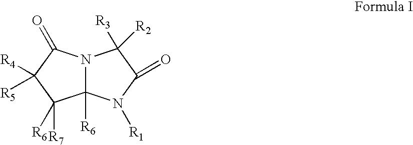
wherein:
R₁-R₈ are individually selected from the group consisting of: H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein any of R₁-R₈ can be further substituted with one or more halogen atoms or functional groups selected from the group consisting of: amides, ureas, ketones, sulfonamides, phosphoramides, alcohols, thiols, esters, amines, amidines, guanidines, carboxylic acids, sulfonic acids, phosphonic acids, and boronic acids;
R₄-R₇ can optionally be side chains with N, S, O, P, or B directly attached to ring of Formula I, substituted with any combination of H, C, C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein the directly attached N, S, or O can also be further attached to a carbonyl, sulfonyl, sulfinyl, or phosphoryl group to form a functional group selected from the group consisting of urea, amide, urethane, sulfonamides sulfmamide, sulfonylurea, sulfinylurea, and phosphoramide, wherein the functional group can be further substituted with H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl, wherein all of the side chains with N, S, O, P, or B can optionally contain one or more halogen atoms and a functional group selected from the group consisting of amide, urea, ketone, sulfonamide, phosphoramide, alcohol, thiol, ester, amine, amidine, guanidine, carboxylic acid, sulfonic acid, phosphonic acid, and boronic acid;
any combination of two substituents selected from R₁-R₈ can be covalently attached to each other to form another ring;
wherein the compound comprises an enantiomer, a diastereomer, or a mixture of the enantiomer and the diastereomer, or a pharmaceutically acceptable salt thereof.

31. A method of preparing a hexahydro-pyrrolo[1,2-a]pyrimidine-2,6-dione derivative compound, said method comprising:
providing a functionalized resin solid support;
reacting the functionalized resin solid support with amino acids under conditions effective to produce a resin bound dipeptide or tripeptide alcohol intermediate compound;
oxidizing the resin bound dipeptide or tripeptide alcohol intermediate compound under conditions effective to convert the resin bound dipeptide or tripeptide alcohol intermediate compound to a resin bound dipeptide or tripeptide aldehyde intermediate compound; and
cyclizing the resin bound dipeptide or tripeptide aldehyde intermediate compound under conditions effective to produce the hexahydro-pyrrolo[1,2-a]pyrimidine-2,6-dione derivative compound.

32. The method according to claim 31, wherein the resin bound dipeptide or tripeptide alcohol intermediate is cleaved from the solid support and said oxidizing and said cyclizing are performed in solution.

33. The method according to claim 32, wherein the resin bound dipeptide or tripeptide alcohol intermediate is cleaved from the solid support by using trifluoroacetic acid/dichloromethane/triethyl silane or trifluoroacetic acid/triisopropylsilane/H₂O.

34. The method according to claim 31, wherein the functionalized resin solid support is an amine resin.

35. The method according to claim 34, wherein the amine resin is prepared by treating a formyl monomethoxy (FMP) resin or a rink amide (MBHA) resin.

36. The method according to claim 34, wherein the amine resin is a substituted amine resin.

37. The method according to claim 34, wherein the amine resin is an unsubstituted amine resin.

38. The method according to claim 31, wherein said reacting involves a first coupling step wherein a first amino acid is coupled to the functionalized resin solid support under conditions effective to produce a resin bound amino acid intermediate compound and a second coupling step wherein a second amino acid is coupled to said resin bound amino acid intermediate compound under conditions effective to produce a resin bound dipeptide alcohol intermediate compound.

39. The method according to claim 38, wherein the first amino acid is a Fmoc-β-alanine.

40. The method according to claim 38, wherein the first coupling step is carried out by using a coupling reagent selected from the group consisting of: O-(7-azabenzotriazol-1-yl)-1,1,3,3-tetramethyluronium hexafluorophosphate, 0-(benzotriazol-1-yl)-1,1,3,3-tetramethyluronium hexafluorophosphate, 0-(benzotriazol-1-yl)-1,1,3,3-tetramethyluronium tetrafluoroborate, 1,3-diisopropylcarbodiimide, 1-(3-dimethylaminopropyl)-3-ethylcarbodiimide hydrochloride, 1-benzotriazolyloxytris(pyrrolidino)phosphonium hexafluorophosphate, and bromotris(pyrrolidino)phosphonium hexafluorophosphate.

41. The method according to claim 40, wherein the coupling reagent is 1,3-diisopropylcarbodiimide or 1-benzotriazolyloxytris(pyrrolidino)phosphonium hexafluorophosphate.

42. The method according to claim 38, wherein the second coupling step is carried out by using a coupling reagent selected from the group consisting of: 0-(7-azabenzotriazol-1-yl)-1,1,3,3-tetramethyluronium hexafluorophosphate, O-(benzotriazol-1-yl)-1,1,3,3-tetramethyluronium hexafluorophosphate, O-(benzotriazol-1-yl)-1,1,3,3-tetramethyluronium tetrafluoroborate, 1,3-diisopropylcarbodiimide, 1-(3-dimethylaminopropyl)-3-ethylcarbodiimide hydrochloride, 1-benzotriazolyloxytris(pyrrolidino)phosphonium hexafluorophosphate, and bromotris(pyrrolidino)phosphonium hexafluorophosphate.

43. The method according to claim 42, wherein the coupling reagent is 1-benzotriazolyloxytris(pyrrolidino)phosphonium hexafluorophosphate.

44. The method according to claim 31, wherein said oxidizing is carried out by using an oxidation reagent selected from the group consisting of:
Swem, pyridine-SO₃, tetra-n-propylammonium perruthenate(VII), pyridinium chlorochromate, pyridinium dichromate, and Dess-Martin periodinane.

45. The method according to claim 44, wherein the oxidation reagent is Dess-Martin periodinane.

46. The method according to claim 31, wherein said cyclizing is carried out under alcohol refluxing conditions.

47. The method according to claim 31, wherein the hexahydro-pyrrolo[1,2-a]pyrimnidine-2,6-dione derivative compound has the following formula: 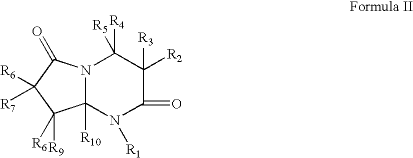
wherein:
R₁-R₁₀ are individually selected from the group consisting of: H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, alkylheteroaryl, and substituted alkylheteroaryl, wherein any of R₁-R₁₀ can be further substituted with one or more halogen atoms or functional groups selected from the group consisting of: amides, ureas, ketones, sulfonamides, phosphoramides, alcohols, thiols, esters, amines, amidines, guanidines, carboxylic acids, sulfonic acids, phosphonic acids, and boronic acids;
R₆-R₉ can optionally be side chains with N, S, O, P, or B directly attached to ring of Formula I, substituted with any combination of H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein the directly attached N, S, or O can also be further attached to a carbonyl, sulfonyl, sulfinyl, or phosphoryl group to form a functional group selected from the group consisting of urea, amide, urethane, sulfonamides sulfinamide, sulfonylurea, sulfinylurea, and phosphoramide, wherein the functional group can be further substituted with H, C, —C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl, wherein all of the side chains with N, S, O, P, or B can optionally contain one or more halogen atoms and a functional group selected from the group consisting of amide, urea, ketone, sulfonamide, phosphoramide, alcohol, thiol, ester, amine, amidine, guanidine, carboxylic acid, sulfonic acid, phosphonic acid, and boronic acid;
any combination of two substituents selected from R₁-R₁₀ can be covalently attached to each other to form another ring;
wherein the compound comprises an enantiomer, a diastereomer, or a mixture of the enantiomer and the diastereomer, or a pharmaceutically acceptable salt thereof.

48. A method of preparing a tetrahydro-pyrrolo[1,2-a]imidazole-2,5-dione derivative compound, said method comprising:
treating amino acids under conditions effective to produce a dipeptide or tripeptide alcohol intermediate compound;
oxidizing the dipeptide or tripeptide alcohol intermediate compound under conditions effective to convert the dipeptide or tripeptide alcohol intermediate compound to a dipeptide or tripeptide aldehyde intermediate compound; and
cyclizing the dipeptide or tripeptide aldehyde intermediate compound under alcohol refluxing conditions to produce the tetrahydro-pyrrolo[1,2-a]imidazole-2,5-dione derivative compound, wherein said treating, said oxidizing, and said cyclizing are all performed in solution.

49. The method according to claim 48, wherein said oxidizing is carried out with Dess-Martin periodinane.

50. The method according to claim 48, wherein the tetrahydro-pyrrolo[1,2-a]imidazole-2,5-dione derivative compound has the following formula: 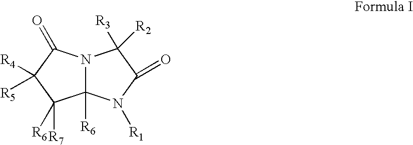
wherein:
R₁-R₈ are individually selected from the group consisting of: H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein any of R₁-R₈ can be further substituted with one or more halogen atoms or functional groups selected from the group consisting of amides, ureas, ketones, sulfonamides, phosphoramides, alcohols, thiols, esters, amines, amidines, guanidines, carboxylic acids, sulfonic acids, phosphonic acids, and boronic acids;
R₄-R₇ can optionally be side chains with N, S, O, P, or B directly attached to ring of Formula I, substituted with any combination of H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein the directly attached N, S, or O can also be further attached to a carbonyl, sulfonyl, sulfinyl, or phosphoryl group to form a functional group selected from the group consisting of urea, amide, urethane, sulfonamides sulfinamide, sulfonylurea, sulfinylurea, and phosphoramide, wherein the functional group can be further substituted with H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl, wherein all of the side chains with N, S, O, P, or B can optionally contain one or more halogen atoms and a functional group selected from the group consisting of amide, urea, ketone, sulfonamide, phosphoramide, alcohol, thiol, ester, amine, amidine, guanidine, carboxylic acid, sulfonic acid, phosphonic acid, and boronic acid;
any combination of two substituents selected from R₁-R₈ can be covalently attached to each other to form another ring;
wherein the compound comprises an enantiomer, a diastereomer, or a mixture of the enantiomer and the diastereomer, or a pharmaceutically acceptable salt thereof.

51. A method of preparing a hexahydro-pyrrolo[1,2-a]pyrimidine-2,6-dione derivative compound, said method comprising:
treating amino acids under conditions effective to produce a dipeptide or tripeptide alcohol intermediate compound;
oxidizing the dipeptide or tripeptide alcohol intermediate compound under conditions effective to convert the dipeptide or tripeptide alcohol intermediate compound to a dipeptide or tripeptide aldehyde intermediate compound; and
cyclizing the dipeptide or tripeptide aldehyde intermediate compound under alcohol refluxing conditions to produce the hexahydro-pyrrolo[1,2-a]pyrimidine-2,6-dione derivative compound, wherein said treating, said oxidizing, and said cyclizing are all performed in solution.

52. The method according to claim 51, wherein said oxidizing is carried out with Dess-Martin periodinane.

53. The method according to claim 51, wherein the hexahydro-pyrrolo[1,2-a]pyrimidine-2,6-dione derivative compound has the following formula: 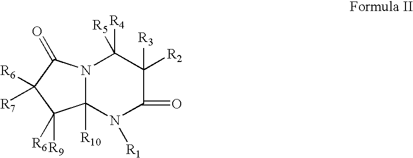
wherein:
R₁-R₁₀ are individually selected from the group consisting of: H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, alkylheteroaryl, and substituted alkylheteroaryl, wherein any of R₁-R₁₀ can be further substituted with one or more halogen atoms or functional groups selected from the group consisting of: amides, ureas, ketones, sulfonamides, phosphoramides, alcohols, thiols, esters, amines, amidines, guanidines, carboxylic acids, sulfonic acids, phosphonic acids, and boronic acids;
R₆-R₉ can optionally be side chains with N, S, O, P, or B directly attached to ring of Formula I, substituted with any combination of H, C₁-C₁₂ alkyl heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, and alkylheteroaryl and substituted alkylheteroaryl, wherein the directly attached N, S, or O can also be further attached to a carbonyl, sulfonyl, sulfinyl, or phosphoryl group to form a functional group selected from the group consisting of urea, amide, urethane, sulfonamides sulfinamide, sulfonylurea, sulfinylurea, and phosphoramide, wherein the functional group can be further substituted with H, C₁-C₁₂ alkyl, heteroatom and multiple heteroatom substituted C₁-C₁₂ alkyl, C₁-C₁₂ branched or cyclic alkyl, heteroatom and multiple heteroatom substituted C₁-C₁₂ branched or cyclic alkyl, aryl and substituted aryl, heteroaryl and substituted heteroaryl, alkylaryl and substituted alkylaryl, or alkylheteroaryl and substituted alkylheteroaryl, wherein all of the side chains with N, S, O, P, or B can optionally contain one or more halogen atoms and a functional group selected from the group consisting of amide, urea, ketone, sulfonamide, phosphoramide, alcohol, thiol, ester, amine, amidine, guanidine, carboxylic acid, sulfonic acid, phosphonic acid, and boronic acid;
any combination of two substituents selected from R₁-R₁₀ can be covalently attached to each other to form another ring;
wherein the compound comprises an enantiomer, a diastereomer, or a mixture of the enantiomer and the diastereomer, or a pharmaceutically acceptable salt thereof.
